(19) United States
(12) Patent Application Publication
(10) Pub. No.: US 2014/0066485 A1
(43) Pub. Date: Mar. 6, 2014

(54) MEDICAMENTS CONTAINING FAMOTIDINE AND IBUPROFEN AND ADMINISTRATION OF SAME

(71) Applicant: Horizon Pharma USA, Inc., (US)

(72) Inventors: George F. Tidmarsh, Portola Valley, CA (US); Barry L. Golombik, Incline Village, NV (US); Lii Tianshiuan, Wilson, NC (US)

(73) Assignee: HORIZON PHARMA USA, INC., Deerfield, IL (US)

(21) Appl. No.: 13/769,196

(22) Filed: Feb. 15, 2013

Related U.S. Application Data
(63) Continuation of application No. 11/489,705, filed on Jul. 18, 2006, now abandoned.
(60) Provisional application No. 60/700,481, filed on Jul. 18, 2005.

(51) Int. Cl. A61K 31/426; A61K 31/192

(52) U.S. Cl. CPC A61K 31/426; A61K 31/192
USPC 514/370

(57) ABSTRACT

A method for administration of ibuprofen to a subject in need of ibuprofen treatment is provided, in which an oral dosage form comprising a therapeutically effective amount of ibuprofen and a therapeutically effective amount of famotidine, in admixture, is administered three times per day.

### 1.0 CROSS-REFERENCE TO RELATED APPLICATIONS

This application claims benefit of U.S. Provisional Application No. 60/700,481, filed Jul. 18, 2005, the entire contents of which are incorporated herein by reference.

### 2.0 FIELD OF THE INVENTION

The invention relates to pharmaceutical compositions containing ibuprofen and famotidine, and finds application in the field of medicine.

### 3.0 BACKGROUND OF THE INVENTION

Ibuprofen, a non-steroidal anti-inflammatory drug (NSAID), has been used in humans for nearly forty years. While generally regarded as safe, ibuprofen and other NSAIDs can cause gastritis, dyspepsia, and gastric and duodenal ulceration. Gastric and duodenal ulceration is a consequence of impaired mucosal integrity resulting from ibuprofen-mediated inhibition of prostaglandin synthesis. This side-effect is a particular problem for individuals who take ibuprofen for extended periods of time, such as patients suffering from rheumatoid arthritis and osteoarthritis.

The risk of developing gastric or duodenal ulceration can be reduced by co-therapy with the drug famotidine. Famotidine blocks the action of the histamine type 2 (H2) receptor, leading to a reduction of acid secretion in the stomach. Reducing stomach acid with famotidine during treatment with certain nonsteroidal anti-inflammatory drugs is reported to decrease incidence of gastrointestinal ulcers (see Taha et al., 1996, “Famotidine for the prevention of gastric and duodenal ulcers caused by nonsteroidal anti-inflammatory drugs” N Engl J Med 334:1435-9, and Rostom et al., 2002, “Prevention of NSAID-induced gastrointestinal ulcers” Cochrane Database Syst Rev 4:CD002296).

Famotidine is used for treatment of heartburn, ulcers, and esophagitis at daily doses from 10 mg to 80 mg. Approved schedules of famotidine administration include 10 or 20 mg QD or BID (for treatment of heartburn), 20 mg or 40 mg QD (for healing ulcers, such as 40 mg HS for 4-8 weeks for healing duodenal ulcers), 20 mg HS (maintenance dose following healing of ulcer), 20 mg BID for 6 weeks (for treatment of gastroesophageal reflux disease), and 20 or 40 mg BID (for treatment of esophageal erosion). For treatment of Zollinger-Ellison Syndrome, a disease characterized by hypersecretion of gastric acid, doses of up to 800 mg/day have been used.

Although NSAID plus famotidine cotherapy reduces risk of developing gastric or duodenal ulceration, present therapies are not widely used. More effective methods of treatment and pharmaceutical compositions are needed. The present invention meets this and other needs.

### 4.0 BRIEF SUMMARY OF THE INVENTION

In one embodiment, ibuprofen and famotidine are administered at total daily doses of about 2400 mg and about 80 mg respectively. In some embodiments of this method, the oral dosage form contains ibuprofen and famotidine in a ratio in the range of 29:1 to 32:1, such as the range of 30:1 to 31:1. In one embodiment, the oral dosage form contains 750 mg to 850 mg (e.g. about 800 mg) ibuprofen and 24 mg to 28 mg (e.g., about 26.6 mg famotidine). In another embodiment, the oral dosage form contains 375 mg to 425 mg (e.g., about 400 mg) ibuprofen and 12 mg to 14 mg (e.g., about 13 mg) famotidine.

In one embodiment, the famotidine and ibuprofen are released from the dosage form rapidly, e.g., under in vitro assay conditions. In one embodiment, the famotidine and ibuprofen are substantially released under low pH conditions.

In one embodiment, the TID administration of the dosage form provides better gastric protection for the subject over a 24-hour period than TID administration of the same daily quantity of ibuprofen and two times a day (BID) administration of the same daily quantity of famotidine. For example, when ibuprofen is administered in the form of an oral dosage form of the invention, the subject's intragastric pH may be greater than 3.5 for at least 18 hours, or even at least 20 hours, of a 24 hour dosing cycle. In one embodiment, the daily quantity of ibuprofen is about 2400 mg and the daily quantity of famotidine is about 80 mg. Thus, in certain aspects, the invention provides a method in which TID administration of a dosage form containing 800 mg ibuprofen and 26.6 mg famotidine provides better gastric protection over a 24-hour period than TID administration of the 800 mg ibuprofen and BID administration of 40 mg famotidine. Equivalently, TID administration of two oral dosage forms containing 400 mg ibuprofen and 13 mg famotidine provides better gastric protection over a 24-hour period than TID administration 800 mg ibuprofen in a single or split dose and BID administration of 40 mg famotidine in a single or split dose.

Ibuprofen, in the form of a unit dose form of the invention, may be administered to a subject is in need of ibuprofen treatment. In various embodiments, the subject is in need of ibuprofen treatment for a chronic condition (such as rheumatoid arthritis, osteoarthritis or chronic pain) or a condition such as acute or moderate pain, dysmenorrhea or acute inflammation.

In one aspect, the present invention is directed to a solid pharmaceutical composition for oral administration which comprises one or more non-steroidal anti-inflammatory (NSAID) compounds, or a pharmaceutically acceptable salt thereof, and famotidine, in admixture with one or more excipients, in a pharmacokinetically effective ratio such that said NSAID(s) and said famotidine are released in a bioequivalent manner.

In a preferred embodiment, the present invention is directed to a solid tablet formulation of ibuprofen or its pharmaceutically acceptable salts, wherein the formulation comprises a therapeutically effective amount of ibuprofen in combination with a therapeutically effective amount of famotidine, with pharmaceutically acceptable excipients in a pharmacokinetically effective ratio, a proportion that allows for specific pharmacokinetic parameters once administered to a subject in need thereof.

In a particular embodiment, the NSAID and famotidine are released from said formulation simultaneously, at a rate and in a ratio providing each in a therapeutically effective and non-toxic amount.

In one embodiment, the compositions of the present invention do not contain any therapeutically active ingredient in addition to one or more NSAID and famotidine.

In a specific embodiment, the NSAID is selected from the group consisting of aspirin, diclofenac, meclofenamate, mefenamic acid, meloxicam, nabumetone, naproxen, oxaprozin, phenylbutazone, piroxicam, sulindac, tenoxicam, diflunisal, tiaprofenic acid, tolmetin, etodolac, fenoprofen, floctafenine, flurbiprofen, ibuprofen, indomethacin, and ketoprofen.

In another embodiment, the pharmaceutical composition is in a unit dose form such as a tablet, pill, capsule, caplet, or gelcap.

The present invention provides a method for administration of ibuprofen to a patient in need of ibuprofen treatment by administering an oral dosage form comprising ibuprofen, famotidine, and pharmaceutically acceptable excipients, three times per day (TID). In one embodiment, the oral dosage form comprises about 800 mg ibuprofen and about 26.6 mg famotidine. In one embodiment, the oral dosage form comprises about 600 mg ibuprofen and about 26.6 mg famotidine. In one embodiment, the oral dosage form comprises about 400 mg ibuprofen and about 13.3 mg famotidine.

In an embodiment the invention provides a solid unit dose form for oral administration which comprises one or more non-steroidal anti-inflammatory (NSAID) compounds, or a pharmaceutically acceptable salt thereof, and famotidine, in admixture with one or more excipients, in a pharmacokinetically effective ratio such that said NSAID(s) and said famotidine are released in a bioequivalent manner.

In an embodiment the pharmaceutical composition of claim 1 comprising ibuprofen and famotidine in the absence of other therapeutically active ingredients.

In an embodiment the ibuprofen and famotidine are released from said formulation simultaneously, at a rate and in a ratio providing each in a therapeutically effective and non-toxic amount.

In an embodiment the pharmaceutical composition comprises 200-800 mg ibuprofen and 20-40 mg famotidine.

In an embodiment the pharmaceutical composition is suitable for administration at least three times per day.

In an embodiment the pharmaceutical composition of claim 1 reducing the gastrointestinal side effects of exerted by said NSAID when administered alone.

In an aspect the present invention provides a method of treating chronic pain, an inflammatory condition, or a condition associated with chronic pain or an inflammatory condition, comprising administering to a subject in need an effective amount of a pharmaceutical composition as described herein.

In an embodiment the method of claim 24 wherein said composition is administered to treat a condition selected from the group consisting of chronic pain, tenderness, inflammation, swelling, fever, headache, or stiffness caused by inflammatory conditions, muscle ache, menstrual pain, injuries, common cold, backache, and surgery or dental work related pain or inflammation.

In an embodiment the inflammatory condition is arthritis or gout.

In an aspect the present invention provides a method for reducing the gastro-intestinal side-effects of a non-steroidal anti-inflammatory compound (NSAID), comprising administering said NSAID as part of a pharmaceutical composition comprising said non-steroidal anti-inflammatory (NSAID) compound, or a pharmaceutically acceptable salt thereof, and famotidine, in the absence of other therapeutically active ingredients, in admixture with one or more excipients, in a pharmacokinetically effective ratio such that said NSAID(s) and said famotidine are released in a bioequivalent manner.

In an aspect the invention provides a method for administration of ibuprofen to a subject in need of ibuprofen treatment, by administering an oral dosage form containing a therapeutically effective amount of ibuprofen and a therapeutically effective amount of famotidine, where the ibuprofen and the famotidine are combined in an admixture with at least one excipient and where the oral dosage form is administered three times per day (TID). In one embodiment the famotidine and ibuprofen are released from the dosage form rapidly in an aqueous environment.

In one embodiment the TID administration of the dosage form of the invention provides better gastric protection over a 24-hour period than TID administration of the same daily quantity of ibuprofen and two times a day (BID) administration of the same daily quantity of famotidine.

In one embodiment the daily quantity of ibuprofen is about 2400 mg and the daily quantity of famotidine is about 80 mg. In one embodiment TID administration of a dosage form of the invention containing 800 mg ibuprofen and 26.6 mg famotidine provides better gastric protection over a 24-hour period than TID administration of the 800 mg ibuprofen and BID administration of 40 mg famotidine. In one embodiment the subject's intragastric pH is greater than 3.5 for at least 18 hours of a 24 hour dosing cycle. In one embodiment the subject's intragastric pH is greater than 3.5 for at least 20 hours of a 24 hour dosing cycle.

In one embodiment the oral dosage form administered according to the method contains ibuprofen and famotidine in a ratio in the range of 29:1 to 32:1, such as a ratio in the range of 30:1 to 31:1. In one embodiment the oral dosage form contains about 750 mg to 850 mg ibuprofen and about 24 mg to 28 mg famotidine. In one embodiment the oral dosage form contains about 375 mg to about 425 mg ibuprofen and about 12 mg to 14 mg famotidine. In one embodiment the oral dosage form contains ibuprofen and famotidine in a ratio in the range of 20:1 to 25:1. In one embodiment the oral dosage form contains ibuprofen and famotidine in a ratio in the range of 22:1 to 23:1. In one embodiment each dosage form contains about 400 mg ibuprofen and about 13.3 mg famotidine. In one embodiment each dosage form contains about 800 mg ibuprofen and about 26.6 mg famotidine. In one embodiment each dosage form contains about 600 mg ibuprofen and about 26.6 mg famotidine. The subject may be in need of ibuprofen treatment for a chronic condition, such as rheumatoid arthritis, osteoarthritis or chronic pain, or a non-chronic condition such as acute pain, dysmenorrhea or acute inflammation.

In one aspect, the invention provides a solid oral dosage form containing a therapeutically effective amount of ibuprofen and a therapeutically effective amount of famotidine, where the ibuprofen and the famotidine are combined in an admixture with at least one excipient, where in an aqueous environment the ibuprofen and famotidine are released into solution rapidly and where the oral dosage form comprises famotidine in the range of 24 mg to 28 mg or in the range 12 mg to 14 mg. In an embodiment, the oral dosage form contains about 13.3 mg famotidine or about 26.6 mg famotidine. In one embodiment the oral dosage form contains ibuprofen and famotidine in a ratio in the range of 29:1 to 32:1 or 22:1 to 23:1. In one embodiment the oral dosage form contains about 800 mg ibuprofen and about 26.6 mg famotidine or about 600 mg ibuprofen and about 26.6 mg famotidine or about 400 mg ibuprofen and about 13.3 mg famotidine.

In some versions of the oral dosage form at least 75% of the famotidine and at least 75% of the ibuprofen in the dosage form are released within 15 minutes when measured in a Type II dissolution apparatus (paddles) according to U.S. Pharmacopoeia XXIX at 37° C. in 50 mM potassium phosphate buffer, pH 7.2 at 50 rotations per minute.

In one embodiment the oral dosage form is a tablet.

In one embodiment, the dosage form contains 60-80% ibuprofen; 1.5-3.0% famotidine; 9-11% microcrystalline cellulose; 2-4% silicified microcrystalline cellulose; and 0.5-2.5% croscarmellose sodium. The formulation may contain 60-80% ibuprofen; 1.5-3.0% famotidine; 9-11% microcrystalline cellulose; 2-4% silicified microcrystalline cellulose; 1-3% low substituted hydroxylpropylcellulose; and 0.5-2.5% croscarmellose sodium.

In one embodiment the formulation comprises ibuprofen, famotidine, microcrystalline cellulose, starch, hydroxypropyl cellulose, low substituted hydroxypropyl cellulose, silicon dioxide, silicified microcrystalline cellulose, croscarmellose sodium and magnesium stearate.

In one embodiment the formulation contains 60-80% ibuprofen; 1.5-3.0% famotidine; 9-11% microcrystalline cellulose; 0.5-1.5% pregelatinized starch (e.g. Starch 1500), 0.2-1% hydroxypropyl cellulose, 1-3% low substituted hydroxypropyl cellulose, 0.2-1% silicon dioxide, 2-4% silicified microcrystalline cellulose; 0.5-2.5% croscarmellose sodium, and 0.5-2.9% magnesium stearate.

In one embodiment the formulation contains 76-78% ibuprofen; 1.5-2.5% famotidine; 9-11% microcrystalline cellulose; 0.5-1.5% pregelatinized starch, 0.2-1% hydroxypropyl cellulose, 1-3% low substituted hydroxypropyl cellulose, 0.2-1% silicon dioxide, 2-4% silicified microcrystalline cellulose; 0.5-2.5% croscarmellose sodium, and 0.5-2.9% magnesium stearate.

In certain embodiments the microcrystalline cellulose is comprised of a first population of particles having a median particle size of about 50 microns (e.g., EMOCEL 50M) and a second population of particles having a median particle size of approximately 90 microns (e.g., EMOCEL 90M). In some embodiments, 50-micron particles are present in at least 10-fold excess, and sometimes at least a 20-fold excess, over 90-micron particles.

In certain embodiments the silicified microcrystalline cellulose (SMCC) is comprised of a first population of particles having a median particle size of about 50 microns (e.g., PROSOLV 50 from Penwest) and a second population of particles having a median particle size of approximately 90 microns (e.g., PROSOLV 90 from Penwest). In one embodiment, the two populations are present in approximately equal quantities.

In one embodiment the oral dosage form contains famotidine (1.5-2.5%); microcrystalline cellulose—median particle size 50 microns (9-10%); pregelatinzed starch (0.8-10%); hydroxypropyl cellulose (0.4-0.8%); ibuprofen (70-80%); colloidal silicon dioxide (0.05-0.10%); microcrystalline cellulose—median particle size 90 microns (0.2-0.6%); silicified microcystalline cellulose—median particle size 50 microns (1-2%); silicified microcrystalline cellulose—median particle size 90 microns (1-2%); low substituted HPC (1-2%); croscarmellose sodium (1-3%) and magnesium stearate (2-2.9%).

In some embodiments the oral dosage form comprises an over-coating layer. In one embodiment the over-coating layer comprises Opadry.

In an aspect, the invention provides a method of treating a patient in need of ibuprofen treatment, where the patient is at elevated risk for developing an NSAID-induced ulcer, containing administering an oral dosage form as described herein.

In an aspect, the invention provides a method for reducing symptoms of dyspepsia in a subject in need of NSAID treatment who has experienced symptoms of dyspepsia associated with NSAID administration, containing administering to the subject an effective amount of a NSAID in combination with an effective amount of famotidine, where the famotidine is administered three times per day. In an embodiment the NSAID is ibuprofen. In one embodiment 25 mg to 27 mg famotidine is administered three times per day.

In an aspect, the invention provides a method of making a tablet containing ibuprofen and famotidine by a) preparing famotidine granules by wet granulating famotidine in the presence of binder and disentegrant and milling and screening the product; b) mixing ibuprofen and a glident to produce an ibuprofen/glident mixture (intermediate mixture I); c) mixing microcrystalline cellulose, silicified microcrystalline cellulose, low substituted HPC, and croscarmellose sodium (intermediate mixture II); d) combining the famotidine granules with intermediate mixture I (ibuprofen/glidant mixture) to produce intermediate mixture III; e) combining intermediate mixture II and intermediate mixture III to produce intermediate mixture IV; f) combining magnesium stearate to intermediate IV, thereby producing a ibuprofen/famotidine solid formulation; and g) compressing the ibuprofen/famotidine solid formulation to form tablets. In some embodiments the famotidine granules in (a) are prepared by combining and blending famotidine, microcrystalline cellulose, pregelatinized starch and hydroxypropyl cellulose, adding water as the granulating liquid, drying the famotidine, and milling and screening the product; and/or (ii) the glident in step (b) is colloidal silicon dioxide.

In an aspect the invention provides a method of making a tablet comprising ibuprofen and famotidine by a) preparing famotidine granules by wet granulating famotidine in the presence of microcrystalline cellulose, starch 1500, and hydroxypropyl cellulose; b) combining microcrystalline cellulose, silicified microcrystalline cellulose, low substituted HPC, and croscarmellose sodium and adding the resulting mixture to the famotidine granules to produce Intermediate Mixture I; c) combining ibuprofen and colloidal silicon dioxide to produce intermediate mixture II; and d) combining Intermediate Mixtures I and II to form a solid formulation containing ibuprofen and famotidine. In some embodiments, the method included compressing the solid formulation to form tablets.

In an aspect, the invention provides ibuprofen and famotidine-containing tablets made according to a method disclosed herein.

### 5.0 BRIEF DESCRIPTION OF THE FIGURES

FIG. 1 shows the predicted effect on intragastric pH of administration of 26.6 mg famotidine TID. FIG. 1A (upper panel) shows the predicted intragastric pH during TID dosing of famotidine (80 mg/day). FIG. 1B (lower panel) shows the predicted plasma famotidine concentration during TID dosing of famotidine (80 mg/day).

FIG. 2 shows the predicted effect on intragastric pH of administration of 40 mg famotidine BID. FIG. 2A (upper panel) shows the predicted intragastric pH during BID dosing of famotidine (80 mg/day). FIG. 2B (lower panel) shows the predicted plasma famotidine concentration during BID dosing of famotidine (80 mg/day).

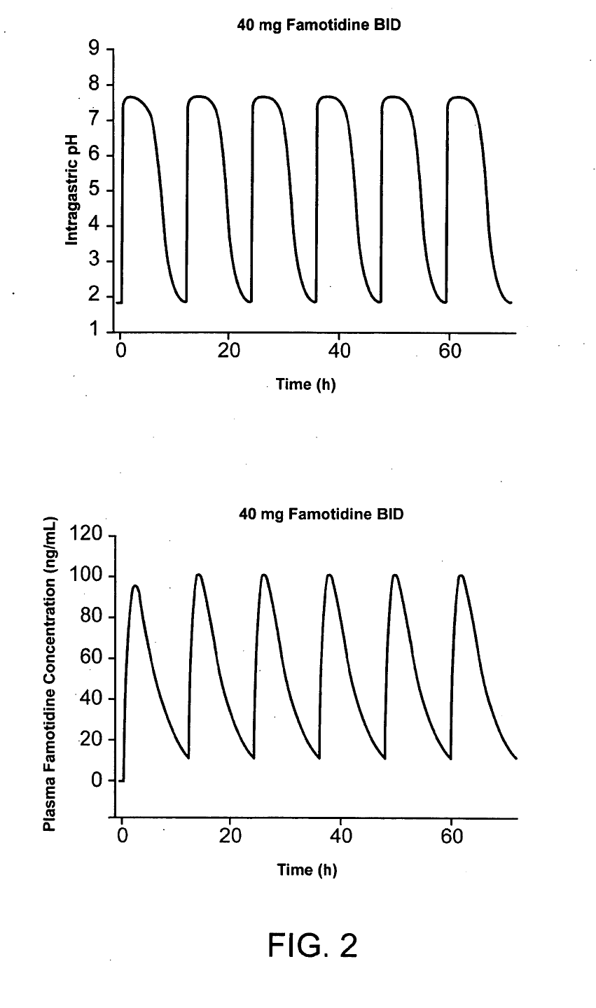

FIG. 3 is a flow chart showing manufacture of unit dose tablets of the invention.

FIG. 4 is a flow chart showing manufacture of unit dose tablets of the invention.

FIG. 5 is a flow chart showing manufacture of unit dose tablets of the invention.

### 6.0 DETAILED DESCRIPTION

### Definitions

6.1 “Famotidine” is 3-[2-(diaminomethyleneamino)thiazol-4-ylmethylthio]-N-sulfamoylpropionamidine, including the polymorphic forms designated Form A and Form B (see, e.g. U.S. Pat. Nos. 5,128,477 and 5,120,850) and their mixtures, as well as pharmaceutically acceptable salts thereof. Famotidine can be prepared using art-known methods, such as the method described in U.S. Pat. No. 4,283,408. Famotidine properties have been described in the medical literature (see, e.g., Echizen et al., 1991, Clin Pharmacokinet. 21:178-94).

6.2 “Ibuprofen” is 2-(p-isobutylphenyl) propionic acid (C₁₃H₁₈O₂), including various crystal forms and pharmaceutically acceptable salts. Two enantiomers of ibuprofen exist. As used herein in the context of solid formulations of the invention, “ibuprofen” refers to a racemic mixture or either enantiomer (with a mixture enriched in the S-enantiomer, or a composition substantially free of the R-enantiomer preferred). Ibuprofen is available commercially and, for example, ibuprofen preparations with mean particle sizes of 25, 38, 50, or 90 microns can be obtained from BASF Aktiengesellschaft (Ludwigshafen, Germany). In one embodiment of the invention, a coated ibuprofen product, such as those described in U.S. Pat. No. 6,251,945 is used. One useful Ibuprofen product is available from BASF under the trade name Ibuprofen DC 85™ Ibuprofen's properties have been described in the medical literature (see, e.g., Davies, 1998, “Clinical pharmacokinetics of ibuprofen. The first 30 years” Clin Pharmacokinet 34:101-54)

6.3 An “API” is an active pharmaceutical ingredient. As used herein, “API” refers to ibuprofen and/or famotidine.

6.4 A “therapeutically effective amount” of ibuprofen is an amount of ibuprofen or its pharmaceutically acceptable salt which eliminates, alleviates, or provides relief of the symptoms for which it is administered.

6.5 A “therapeutically effective amount” of famotidine is an amount of famotidine or its pharmaceutically acceptable salt which suppresses gastric acid secretion.

6.6 The terms “solid oral dosage form,” “oral dosage form,” “unit dose form,” “dosage form for oral administration,” and the like are used interchangably, and refer to a pharmaceutical composition in the form of a tablet, capsule, caplet, gelcap, geltab, pill and the like.

6.7 An “excipient,” as used herein, is any component of an oral dosage form that is not an API. Excipients include binders, lubricants, diluents, disintegrants, coatings, glidants, and other components. Excipients are known in the art (see HANDBOOK OF PHARMACEUTICAL EXCIPIENTS, FIFTH EDITION, edited by Rowe et al., McGraw Hill). Some excipients serve multiple functions or are so-called high functionality excipients. For example, talc may act as a lubricant, and an anti-adherent, and a glidant. See Pifferi et al., 2005, “Quality and functionality of excipients” Farmaco. 54:1-14; and Zeleznik and Renak, Business Briefing: Pharmagenerics 2004.

6.8 A “binder” is an excipient that imparts cohesive qualities to components of a pharmaceutical composition. Commonly used binders include, for example, starch; sugars, such as, sucrose, glucose, dextrose, and lactose; cellulose derivatives such as powdered cellulose, microcrystalline cellulose, silicified microcrystalline cellulose (SMCC), hydroxypropylcellulose, low-substituted hydroxypropylcellulose, hypromellose (hydroxypropylmethylcellulose); and mixtures of these and similar ingredients.

6.9 A “lubricant” is an excipient added to reduce sticking by a solid formulation to the equipment used for production of a unit does form, such as, for example, the punches of a tablet press. Examples of lubricants include magnesium stearate and calcium stearate. Other lubricants include, but are not limited to, aluminum-stearate, PEG 4000-8000, talc, sodium benzoate, glyceryl mono fatty acid (e.g. glyceryl monostearate from Danisco, UK), glyceryl dibehenate (e.g. CompritolATO888™ Gattefosse France), glyceryl palmito-stearic ester (e.g. Precirol™, Gattefosse France), polyoxyethylene glycol (PEG, BASF), hydrogenated cotton seed oil or castor seed oil (Cutina H R, Henkel) and others.

6.10 A “diluent” is an excipient added to a pharmaceutical composition to increase bulk weight of the material to be formulated, e.g. tabletted, in order to achieve the desired weight.

6.11 The term “disintegrant” refers to excipients included in a pharmaceutical composition in order to ensure that the composition has an acceptable disintegration rate in an environment of use. Examples of disintegrants include starch derivatives (e.g., sodium carboxymethyl starch and pregelatinized corn starch such as starch 1500 from Colorcon) and salts of carboxymethylcellulose (e.g., sodium carboxymethylcellulose), crospovidone (cross-linked PVP polyvinylpyrrolidinone (PVP), e.g., Polyplasdone™ from ISP or Kollidon™ from BASF).

6.12 The term “glidant” is used to refer to excipients included in a pharmaceutical composition to keep the component powder flowing as the tablet is being made, preventing formation of lumps. Nonlimiting examples of glidants are colloidal silicon dioxides such as CAB-O-SIL™ (Cabot Corp.), SYLOID™, (W.R. Grace & Co.), AEROSIL™ (Degussa) talc, and corn starch.

6.13 The term “nonionic surfactant” refers to, for example and not limitation, sucrose esters; partial fatty acid esters of polyhydroxyethylenesorbitan, such as polyethylene glycol(20) sorbitan monolaurate, monopalmitate, monostearate and monooleate; polyethylene glycol(20) sorbitan tristearate and trioleate); polyethylene glycol(4) sorbitan monolaurate and monostearate; polyethylene glycol(5) sorbitan monooleate; polyhydroxyethylene fatty alcohol ethers such as polyoxyethylene cetyl stearyl ether or corresponding lauryl ethers; polyhydroxyethylene fatty acid esters; ethylene oxide/propylene oxide block copolymers; sugar ethers and sugar esters; phospholipids and their derivatives; and ethoxylated triglycerides such as the derivatives of castor oil. Examples include Cremophor™ RH 40; Cremophor™ RH 60, Tween™ 80.

6.14 The term “over-coating,” “over-coating layer,” or “over-coat” refer to the outer most coating or coatings of a unit dose form such as a tablet or caplet, which may be added to improve appearance, taste, swallowability, or other characteristics of the tablet, caplet, capsule, gelcap, etc. The over coating layer does not contain an API. Suitable over-coatings are soluble in, or rapidly disintegrate in water, and, for purposes of this invention, are not enteric coatings. An exemplary over-coating material is Opadry II available from Colorcon, Inc., Westpoint Pa. Materials for making over-coating layer are well known in the art and include, for example and not limitation, materials are described in U.S. Pat. No. 4,543,370 (Colorcon), incorporated herein by reference. In one embodiment the over coating comprises a non-toxic edible polymer, edible pigment particles, an edible polymer plasticizer, and a surfactant. A preferred material, “Opadry II” is available from Colorcon (West Point Pa. USA) and comprises HPMC, titanium dioxide, plasticizer and other components.

6.15 “QD”, “BID”, “TID”, “QID”, and “HS” have their usual meanings of, respectively, administration of medicine once per day, twice per day, three times per day, four times per day or at bedtime. Administration three times per day means that at least 6 hours, preferably at least 7 hours, and more preferably about 8 hours elapse between administrations. Administration three times per day can mean administration about every 8 hours (e.g., 7 a.m., 3 p.m. and 11 p.m.). In some cases in which quantitative measurements are made, “TID administration” can mean administration every 8±0.25 hours.

6.16 As used herein, the term “daily quantity” refers to the quantity of an API (ibuprofen or famotidine) administered over a 24-hour period under a specific dosing regimen.

6.17 A “subject in need of ibuprofen treatment” is an individual who receives therapeutic benefit from administration of ibuprofen. Ibuprofen is indicated for treatment of mild to moderate pain, dysmenorrhea, inflammation, and arthritis. In one embodiment, the subject in need of ibuprofen treatment is under treatment for a chronic condition. For example and without limitation, a subject in need of ibuprofen treatment may be an individual with rheumatoid arthritis, an individual with osteoarthritis, an individual suffering from chronic pain (e.g., chronic low back pain, chronic regional pain syndrome, chronic soft tissue pain), or an individual suffering from a chronic inflammatory condition. In general, a subject under treatment for a chronic condition requires ibuprofen treatment for an extended period, such as at least one month, at least four months, at least six months, or at least one year. In another embodiment, the subject in need of ibuprofen treatment is under treatment for a condition that is not chronic, such as acute pain, dysmenorrhea or acute inflammation. Preferably the patient in need of ibuprofen treatment does not suffer from a condition characterized by hypersecretion of gastric acid (e.g., Zollinger-Ellison Syndrome). Preferably the patient does not suffer from Barrett's ulceration or active severe oesophagitis. In certain embodiments the subject does not have gastroesophageal reflux disease (GERD). In certain embodiments the subject is not in need of treatment for an ulcer. In certain embodiments the subject does not suffer from dyspepsia. In certain embodiments the subject is at elevated risk of developing an NSAID-induced ulcer.

6.18 An “ibuprofen responsive condition” is a condition for which symptoms are reduced by administration of ibuprofen, such as mild to moderate pain, dysmenorrhea, inflammation, arthritis (e.g., rheumatoid arthritis and osteoarthritis), chronic pain, chronic inflammatory condition, chronic pain, acute pain and acute inflammation.

6.19 A subject is “at elevated risk for developing an NSAID-induced ulcer” if the subject in more susceptible than the average individual to develop an ulcer when under treatment with an NSAID. A high odds ratio for risk of development of NSAID-associated ulcer complications is seen in individuals with a past complicated ulcer (odds ratio 13.5), individuals taking multiple NSAIDs or NSAIDs plus aspirin (odds ratio 9.0); individuals taking high doses of NSAIDs (odds ratio 7.0), individuals under anticoagulant therapy, such as low dose aspirin (odds ration 6.4), individuals with a past uncomplicated ulcer (odds ratio 6.1), and individuals older than 70 years (odds ratio 5.6) See, e.g., Gabriel et al., 1991, Ann Intern Med. 115:787; Garcia Rodriguez et al. 1994, Lancet 343:769; Silverstein et al. 1995, Ann Intern Med. 123:241; and Sorensen et al., 2000, Am J. Gastroenterol. 95:2218. Subjects at increased risk for developing an NSAID-induced ulcer may have one or more of these risk factors. Subjects “at high risk for developing an NSAID-induced ulcer” are individuals older than 80 years of age and subjects with a history of NSAID-associated serious gastrointestinal complications (perforation of ulcers, gastric outlet obstruction due to ulcers, gastrointestinal bleeding).

6.20 “Admixture” refers to a pharmaceutical composition made by combining and mixing two or more drugs and one or more excipients in the same compartment of the unit dosage form.

6.21 As used herein in the context of a unit dosage form, the term “enteric” has its usual meaning and refers to a medicinal preparation that passes through the stomach intact and disintegrates in the intestines. An “enteric coating” remains insoluble at gastric pH, then allows for release of the active ingredient from a coated particle or coated dosage form at pH greater than about 5.0, e.g. 5.5, 6.0, 6.5, or 7.0

6.22 As used herein, “dyspepsia” refers to upper abdominal pain or discomfort with or without symptoms of early satiety, nausea, or vomiting with no definable organic cause, as diagnosed following the Rome II criteria (Talley et al., 1999, Gut 45 (Suppl. II):1137-42), or any subsequent modification thereof. According to the Rome II criteria, a diagnosis of functional dyspepsia requires: (1) persistent or recurrent abdominal pain or discomfort centered in the upper abdomen; (2) symptom duration of at least 12 weeks, which need not be consecutive, within the preceding 12 months; (3) no evidence of organic disease (including at upper endoscopy) that is likely to explain symptoms; (4) no evidence that dyspepsia is exclusively relieved by defecation or association with the onset of a change in the stool frequency or stool form (i.e., not irritable bowel syndrome). In this context, “discomfort” is defined as an unpleasant sensation, and may include fullness, bloating, early satiety, and nausea. The definition includes, without limitation, ulcer-like, dysmotility-like, and non-specific dyspepsia. Symptoms of dyspepsia include nausea, regurgitation, vomiting, heartburn, prolonged abdominal fullness or bloating after a meal, stomach discomfort or pain, and early fullness.

6.24 A unit dose form is in an “aqueous environment” when it is in a water-based solution in vivo (e.g., in the stomach) or in vitro. One in vitro aqueous environment is 50 mM potassium phosphate buffer, pH 7.2. Another in vitro aqueous environment is 50 mM potassium phosphate buffer, pH 4.5.

6.25 By “pharmacokinetically effective ratio” is meant an amount of each of the excipients in relation to one another such that the solid formulation dissolves upon administration to a patient in need of this formulation at a rate and in a manner that the NSAID (e.g., ibuprofen) and the famotidine enter the blood in a manner such that each of these components is bioequivalent to that component when administered as an approved formulation.

6.26 “Bioequivalence” is defined as a pharmacokinetic (PK) comparison of the proposed drug formulation (the formulation of the present invention) to that of the approved formulation. The proposed drug formulation must display drug pharmacokinetics that fall within a range of 80-125% (0.8-1.25) when one computes the ratio of the drug PK when administered as the approved formulation to that when administered as the drug formulation of the present invention. The PK parameters that are used for this comparison are the maximum concentration achieved in the blood (Cmax) and the area-under-the-curve (AUC). The AUC is determined by plotting the concentration of the active ingredient in the blood over time. It is accepted that if the proposed drug formulation (the formulation of the present invention) PK falls within the 80-125% range when compared to the approved drug formulation PK, the proposed drug formulation will have all of the safety and efficacy of the approved drug. The Cmax and AUC determine the activity and side effects of the drug.

6.27 By “pharmacokinetically effective ratio” is meant an amount of each of the excipients in relation to one another such that the solid formulation dissolves upon administration to a patient in need of this formulation at a rate and in a manner that the NSAID and the famotidine enter the blood in a manner such that each of these components is bioequivalent to that component when administered as an approved formulation.

6.28 “Non-steroidal anti-inflammatory drugs” or NSAIDs and various pharmaceutically acceptable salts are described in published literature, the contents of several are incorporated by reference. Examples of NSAIDs include aspirin, diclofenac, meclofenamate, mefenamic acid, meloxicam, nabumetone, naproxen, oxaprozin, phenylbutazone, piroxicam, sulindac, tenoxicam, diflunisal, tiaprofenic acid, tolmetin, etodolac, fenoprofen, floctafenine, flurbiprofen, ibuprofen, indomethacin, and ketoprofen.

6.29 By “therapeutically effective amount” of NSAID is meant that amount of the NSAID or its pharmaceutically acceptable salt which eliminates, alleviates, or provides relief of the symptoms for which the NSAID is administered. The therapeutically effective amount of a drug (e.g., famotidine, ibuprofen, or other NSAID) is determined by an ordinarily skilled artisan, taking into account various considerations, such as the age or the weight of the subject, the condition of the patient, the regimen, the severity of the condition(s) to be treated, the desired result, and the like.

6.30 All percentages are % w/w, unless specifically indicated otherwise. Unless otherwise specified, “% weight” is percent weight of the specified component compared to the total weight of the unit dosage (e.g., tablet) exclusive of any over-coating layer. Optionally the % weight can be calculated based on the total weight of the unit dosage form including the over-coating layer. “United States Pharmacopeia” and “USP” mean the United States Pharmacopeia and National Formulary 29th Revision (available from 12601 Twinbrook Parkway, Rockville, Md. 20852-1790, USA). It will be appreciated that due to round or practical limits on quantitive measurements, reference to a quantity of API or excipient in a dosage form can include some variation, such as ±10%, preferably ±5%, and more preferably ±1%. It will be appreciated, for example, that a total quantity of 80 mg famotidine can be administered in three doses of 26.6 mg famotidine per dose.

### 7.0 TID ADMINISTRATION OF IBUPROFEN-FAMOTIDINE ORAL DOSAGE FORM

In one aspect the present invention relates to administration of an oral dosage form comprising ibuprofen, famotidine, and one or more pharmaceutically acceptable excipients, to a patient in need of ibuprofen treatment. In a particular embodiment, the pharmaceutical composition of the invention is suitable for administration at least three times per day.

Famotidine is currently approved for and generally used on a once or twice per day schedule for prevention of minor gastric irritation. When administered to avoid or mitigate the ulcerogenic effects of long-term NSAID therapy, famotidine is administered at 40 mg BID (see Taha et al., 1996, supra). However, it has now been determined using pharmacokinetic modeling (see Example 1) that, surprisingly, TID administration of famotidine provides a protective effect superior to that achieved by BID dosing. For example, TID administration of famotidine results in intragastric pH higher than 3.5 for a greater proportion of the dosing cycle than conventional BID dosing.

In addition, a human clinical study described in Example 3, below, has shown that the pharmocokinetic parameters for concurrent administration of immediate release forms of ibuprofen and famotidine were not significantly different from pharmocokinetic parameters for separate administration of the two APIs. When administered concurrently, both ibuprofen and famotidine retain immediate release characteristics of rapid absorption and rapid attainment of the maximum plasma concentration (Tₘₐₓ).

These data indicate that a treatment paradigm in which ibuprofen and famotidine are administered as a unit dose form on a TID (three times per day) schedule will deliver ibuprofen that is bioequivalent to that of conventional TID dosing of ibuprofen, while providing significant and superior protection from ibuprofen-related side effects such as increased likelihood ulcer development and dyspepsia. Administration of ibuprofen-famotidine TID will provide superior protection, as measured by gastric pH, compared to cotherapy with famotidine QD and ibuprofen TID.

Thus, in one aspect, the present invention provides a method for administration of ibuprofen to a patient in need of ibuprofen treatment by administering an oral dosage form comprising a therapeutically effective amount of ibuprofen and a therapeutically effective amount of famotidine, wherein the oral dosage form is administered three times per day (TID). The invention also provides oral unit dosage forms adapted for use in this method.

### 8.0 INCOMPATIBILITY OF IBUPROFEN AND FAMOTIDINE

Forced degradation of stress assays are used to evaluate the stability of pharmaceutical compositions. Forced degradation conditions refer to conditions of elevated temperature, or elevated temperature and humidity, intended to accelerate the process of chemical degradation. Forced degradation conditions for a period of time are used to predict the effect of storage under more benign conditions (e.g., room temperature) for a longer period of time.

It has been discovered that, under “forced degradation” conditions, ibuprofen and famotidine in admixture are pharmaceutically incompatible. As shown in Example 4, below, famotidine alone is stable when stored for 2 weeks at 60° C., but is degraded when stored as a mixture with ibuprofen for 2 weeks at 60° C. or for 1 month at 40° C. and 75% relative humidity. Similarly, famotidine degradation is seen when a famotidine-ibuprofen admixture in the form of a tablet is stored 1 month at 60° C. (see Example 5). Surprisingly, however, the tablet form is stable at room temperature for at least 4 months. This suggests that contrary to the conclusion that would be drawn from conventional stress testing, ibuprofen-famotidine tablets according to the invention are stable for a prolonged period under normal storage conditions.

### 9.0 IBUPROFEN-FAMOTIDINE ORAL DOSAGE FORMS: API CONTENT, DISSOLUTION PROPERTIES AND PROTECTIVE PROPERTIES

Exemplary formulations that may be used in the practice of the invention are described below.

9.1 API Content

The dosage forms of the invention comprise ibuprofen and famotidine in amounts sufficient to provide therapeutic efficacy when administered three times per day. At each administration time, a single unit dosage form (e.g., tablet) may be administered, or the appropriate amount of drug can be administered as a split dose (i.e., the same amount of drug administered as two tablets taken together). For example, TID administration of 800 mg ibuprofen and 26.6 mg famotidine can be in the form of a single unit dosage form containing 800 mg ibuprofen and about 26.6 mg famotidine, two unit dosage forms containing 400 mg ibuprofen and about 13.3 mg famotidine, or even four unit dosage forms containing 200 mg ibuprofen and about 7 mg famotidine. Preferably, a therapeutically effective dose is administered as one or two tablets.

Preferably, a therapeutically effective amount of ibuprofen or salt thereof ranges from about 200 mg/day to about 3200 mg/day and more preferably from about 1200 mg/day to about 2400 mg/day. Preferably, a solid tablet formulation contains ibuprofen or its pharmaceutically acceptable salts in an amount ranging from about 20 mg/tablet to about 1600 mg/tablet and more preferably from about 200 mg/tablet to about 800 mg/tablet and, most preferably, from about 400 mg/tablet to about 800 mg/tablet. The therapeutically effective amount of ibuprofen so administered is usually in the range 50 mg to 1000 mg. A therapeutically effective dose for headache or mild pain may be 200 mg or 400 mg TID. A therapeutically effective dose for arthritis is usually 800 mg TID.

In general, the unit dosage forms of the invention comprise ibuprofen in an amount of about 50-1000 mg, such as 50-800 mg. In certain embodiments the unit dosage form comprises ibuprofen in an amount of about 200-800 mg, about 200-400 mg, about 300-500 mg, about 700-800 mg, about 400 mg or about 800 mg ibuprofen.

For many applications the quantity of ibuprofen in the unit dose form is about 800 mg (e.g., in the range 750 mg to 850 mg) which allows administration of 2400 mg/day with TID administration of one tablet, or the quantity of ibuprofen is about 400 mg (e.g., in the range 375 mg to 425 mg) which allows administration of 2400 mg/day with TID administration of two tablets.

Preferably, a solid tablet formulation contains famotidine in an amount ranging from about 5 mg/tablet to about 80 mg/ml and more preferably from about 10 mg/tablet to about 40 mg/tablet and, most preferably, from about 10 mg/tablet to about 20 mg/tablet.

The therapeutically effective amount of famotidine so administered is usually in the range 7 mg to 30 mg. In general, the unit dosage forms of the invention comprise famotidine in the range of 12 mg to 28 mg. For many applications the quantity of famotidine in the unit dose form is about 26.6 mg (e.g., in the range 24 mg to 28 mg) which allows administration of 80 mg/day with TID administration of one tablet, or the quantity of famotidine is about 13 mg (e.g., in the range 12 mg to 14 mg) which allows administration of 80 mg/day with TID administration of two tablets. In another embodiment, the pharmaceutical composition comprises 5-40 mg famotidine, or 10-40 mg famotidine, or 20-40 mg famotidine, or about 10 mg of famotidine, or about 20 mg of famotidine.

In one preferred embodiment, the oral unit dosage forms are formulated to deliver a daily dose of about 2400 mg ibuprofen and about 80 mg famotidine with three times per day administration. For many applications the quantity of ibuprofen is about 800 mg (e.g., in the range 750 mg to 850 mg) and the quantity of famotidine is about 26.6 mg (e.g., in the range 24 mg to 28 mg). This allows administration of 2400 mg/day ibuprofen and 80 mg/day famotidine with TED administration of one tablet. In a related embodiment, the quantity of ibuprofen is about 400 mg (e.g., in the range 375 mg to 425 mg) and the quantity of famotidine is about 13 mg (e.g., in the range 12 mg to 14 mg). This allows administration of 2400 mg/day ibuprofen and 80 mg/day famotidine with TID administration of two tablets. In a related embodiment, the quantity of ibuprofen is about 200 mg (e.g., in the range 175 mg to 225 mg) and the quantity of famotidine is about 6.6 mg (e.g., in the range 6 mg to 7 mg). In yet another embodiment, the invention concerns a pharmaceutical composition comprising about 400 mg ibuprofen and about 10 mg famotidine. In a further embodiment, the invention concerns a pharmaceutical composition comprising about 800 mg ibuprofen and about 20 mg famotidine.

In other embodiments more or less API may be administered. For example, in some cases the daily dose of ibuprofen is greater than 2400 mg (e.g., 3200 mg). This amount can easily be administered as, for example, three or six tablets per day, particularly using an ibuprofen formulation that can be tabletted with little excipient (e.g., BASF Ibuprofen DC 85®). If a formulation that contains only the active S-enantiomer of ibuprofen is used, a smaller quantity may sometimes be administered, such as about half as much as described hereinabove.

In certain embodiments the ratio of ibuprofen to famotidine in the dosage forms of the invention is in the range of 15:1 to 40:1, more often 20:1 to 40:1 and even more often 25:1 to 35:1. In some embodiments the ratio of ibuprofen to famotidine in the dosage forms of the invention is in the range of 29:1 to 32:1, such as 30:1 to 31:1. In one embodiment the ratio of ibuprofen to famotidine is about 30:1. Exemplary amounts of ibuprofen and famotidine include 800±10% mg ibuprofen and 26.6±10% mg famotidine; 600±10% mg ibuprofen and 19.95±10% mg famotidine; 400±10% mg ibuprofen and 13.3±10% mg famotidine; and 200±10% mg ibuprofen and 6.65±10% mg famotidine.

In certain embodiments the ratio of ibuprofen to famotidine in the dosage forms of the invention is in the range of range of 20:1 to 25:1, such as 22:1 to 23:1. In one embodiment the ratio of ibuprofen to famotidine is about 22.5:1. Exemplary amounts of ibuprofen and famotidine include 600±10% mg ibuprofen and 26.6±10% mg famotidine.

In a preferred embodiment, the oral dosage form does not contain a pharmaceutically active compound (i.e., drug compound) other than ibuprofen and famotidine. In particular embodiments the oral dosage form does not contain any NSAID other than ibuprofen and/or does not contain any H2-receptor antagonist other than famotidine. In certain embodiments the amount of famotidine is other than 5 mg, other than 10 mg, other than 20 mg or other than 40 mg per dosage form.

9.2 Rapid Release of Famotidine and Ibuprofen

In a particular embodiment, the NSAID and famotidine are released from the formulation simultaneously, at a rate and in a ratio providing each in a therapeutically effective and non-toxic amount. Thus, oral dosage forms of the invention are formulated so that release of both APIs occurs (or begins to occur) at about the same time. That is, the dosage form is not designed so that one of the APIs is released significantly later than the other API.

In an embodiment the unit dosage form is formulated so that famotidine and ibuprofen are released rapidly under neutral pH conditions (e.g., an aqueous solution at about pH 6.8 to about pH 7.4). In this context “rapidly” means that both APIs are significantly released into solution within 20 minutes under in vitro assay conditions. In some embodiments both APIs are significantly released into solution within 15 minutes under in vitro assay conditions. In this context, “significantly released” means that at least about 60% of the weight of the API in the unit dosage form is dissolved, preferably at least about 75%, more preferably at least about 80%, often at least 90%, and sometimes at least about 95%.

Dissolution rates may be determined using the known methods. Generally an in vitro dissolution assay is carried out by placing the famotidine-ibuprofen unit dosage form(s) (e.g., tablet(s)) in a known volume of dissolution medium in a container with a suitable stirring device. Samples of the medium are withdrawn at various times and analyzed for dissolved active substance to determine the rate of dissolution. Dissolution may be measured as described for ibuprofen in the USP or, alternatively, as described for famotidine in the USP. One approach is illustrated in Example 6. Briefly, the unit dose form (e.g., tablet) is placed in a vessel of a United States Pharmacopeia dissolution apparatus II (Paddles) containing 900 ml dissolution medium at 37° C. The paddle speed is 50 RPM. Independent measurements are made for at least three (3) tablets. In one suitable in vitro assay, dissolution is measured using a neutral dissolution medium such as 50 mM potassium phosphate buffer, pH 7.2 (“neutral conditions”) generally as described in Example 6, below.

For illustration and not limitation Example 6, below, shows dissolution assays carried out using a tablet prepared in accordance with the invention.

9.3 Substantial Release of Famotidine and Ibuprofen Under Low pH Conditions

In an embodiment the unit dosage form is formulated so that famotidine and ibuprofen are both released rapidly under low pH conditions. Release under low pH conditions is measured using the assay described above and in Example 5, but using 50 mM potassium phosphate buffer, pH 4.5 as a dissolution medium. As used in this context, the APIs are released rapidly at low pH when, a substantial amount of both APIs is released into solution within 60 minutes under low pH assay conditions. In some embodiments, a substantial amount of both APIs is released into solution within 40 minutes under low pH assay conditions. In some embodiments, a substantial amount of both APIs is released into solution within 20 minutes under low pH assay conditions. In some embodiments, a substantial amount of both APIs is released into solution within 10 minutes under low pH assay conditions. In this context, a “substantial amount” means at least 15%, preferably at least 20%, and most preferably at least 25% of ibuprofen is dissolved and at least 80%, preferably at least 85%, and most preferably at least 90% of famotidine is dissolved.

For illustration and not limitation Example 6, below, shows dissolution assays carried out using a tablet prepared in accordance with the invention.

9.4 Gastric Protection

As illustrated in Example 1, TID administration to a subject of famotidine results in an intragastric pH that is elevated relative to the intragastric pH resulting from conventional BID administration of famotidine, resulting in better gastric protection. As used herein administration of a pharmaceutical composition or compositions “provides better gastric protection” compared to administration of a reference composition or compositions when administration of the pharmaceutical composition maintains stomach pH at a more basic level. It has now been discovered that TID administration of famotidine provides better gastric protection than conventional BID dosing of the same daily dose of drug.

One measure of gastric protection is the fraction of a 24-hour dosing cycle during which amount of time pH is maintained above a designated value (e.g., pH 3.0, sometimes pH 3.5, sometimes pH 4.0, and sometimes pH 4.5). For example, better gastric protection can be characterized as pH above the designated value for more time (e.g., 20 hours in a 24 hour period vs. 15 hours in a 24 hour period) than administration of the reference composition(s). In one embodiment, TID administration of famotidine (or, alternatively a unit dosage form of the invention containing famotidine and ibuprofen) will maintain a gastric pH of 3.5 or greater for at least 16, at least 17, at least 18, at least 19, at least 20, at least 21, at least 22, or at least 23 hours of a 24 hour dosing cycle. In one embodiment, TID administration of famotidine (or, alternatively a unit dosage form of the invention containing famotidine and ibuprofen) will maintain a gastric pH of 3.0 or greater for at least 16, at least 17, at least 18, at least 19, at least 20, at least 21, at least 22, or at least 23 hours of a 24 hour dosing cycle. In one embodiment, TID administration of famotidine (or, alternatively a unit dosage form of the invention containing famotidine and ibuprofen) will maintain a gastric pH of 3.5 or greater for at least 16, at least 17, at least 18, at least 19, at least 20, at least 21, at least 22, or at least 23 hours of a 24 hour dosing cycle. In one embodiment, TID administration of famotidine (or, alternatively a unit dosage form of the invention containing famotidine and ibuprofen) will maintain a gastric pH of 4.0 or greater for at least 16, at least 17, at least 18, at least 19, at least 20, at least 21, at least 22, or at least 23 hours of a 24 hour dosing cycle. TID administration of famotidine (or, alternatively a unit dosage form of the invention containing famotidine and ibuprofen) will maintain a gastric pH of 4.5 or greater for at least 16, at least 17, at least 18, at least 19, at least 20, at least 21, at least 22, or at least 23 hours of a 24 hour dosing cycle. In one embodiment of the present invention, TID administration of famotidine (or, alternatively TID administration a unit dosage form of the invention containing famotidine and ibuprofen) results in a gastric pH above a specified value (e.g., at least 3.0, at least 3.5, at least 4.0 or at least 4.5) for more hours in a 24-hour dosing cycle that than BID administration of the same daily dose of famotidine (or, alternatively a BID administration of the same daily dose of famotidine and TID administration of the same daily dose of ibuprofen) where the difference in hours is at least 1, at least 2, at least 3, at least 4, or at least 5.

Another measure of gastric protection is the minimum sustained gastric pH during a 24-hour dosing cycle. “Sustained pH” refers to a gastric pH (or pH range) sustained for at least 10 minutes. Better gastric protection can be characterized as a higher minimum sustained pH when measured over a 24-hour dosing period. In one embodiment of the present invention, TID administration of famotidine (or, alternatively a unit dosage form of the invention containing famotidine and ibuprofen) results in a minimum sustained pH of at least 2.0, preferably at least 2.3, more preferably at least 2.5, and sometimes at least 3.0. In one embodiment of the present invention, TID administration of famotidine (or, alternatively TID administration a unit dosage form of the invention containing famotidine and ibuprofen) results in a minimum sustained pH that is higher than BID administration of the same daily dose of famotidine (or, alternatively a BID administration of the same daily dose of famotidine and TID administration of the same daily dose of ibuprofen) where the difference in pH is at least 0.2, at least 0.4, at least 0.5, at least 0.6, or at least 0.7 pH units.

Another measure of gastric protection is the average or median gastric pH during a 24-hour dosing cycle. Better gastric protection can be characterized as a higher average or median gastric pH over a 24-hour dosing period. In one embodiment of the present invention, TID administration of famotidine (or, alternatively a unit dosage form of the invention containing famotidine and ibuprofen) results in an average or median gastric pH of at least 6.0, preferably at least 6.1, more preferably at least 6.2, even more preferably at least 6.3 and sometimes at least 6.4. In one embodiment of the present invention, TID administration of famotidine (or, alternatively TID administration a unit dosage form of the invention containing famotidine and ibuprofen) results in an average or median gastric pH that is higher than BID administration of the same daily dose of famotidine (or, alternatively a BID administration of the same daily dose of famotidine and TED administration of the same daily dose of ibuprofen) where the difference in pH is at least 0.2, at least 0.3, at least 0.4, at least 0.6, at least 0.7 or at least 0.8 pH units.

For illustration, TED administration of a unit dosage form containing 800 mg ibuprofen and 26.6 mg famotidine would provide superior gastric protection than does TID administration of a unit dosage form containing 800 mg ibuprofen and BID administration of a unit dosage form containing 40 mg famotidine.

Intragastric pH can be determined by art-known methods using, for example, a nasogastric pH probe. One useful probe is the Digitrapper™ pH 400 ambulatory pH recorder from Medtronic Functional Diagnostics (Shoreview, Minn.). Measurements can be made after the subject has received the appropriate dosage regimen for 3 days, which allows steady state levels of drug to be achieved.

### 10.0 EXEMPLARY UNIT DOSE FORMS

Unit dose forms of the invention comprise ibuprofen (or other NSAID) in admixture with famotidine and at least one excipient. The unit dose form may be a tablet, caplet, gelcap, or other form. In some embodiments the dosage form includes a core comprising the ibuprofen and famotidine, which core is surrounded by an over coating which may be added to improve appearance, taste, swallowability, or other characteristics of the dosage form. It is preferred that the solid formulation of the present invention is durable to usual external manipulation yet able dissolve at the acceptable rate.

In one preferred embodiment, the solid tablet carrier contains at least one, and preferably at least two, of the following components: microcrystalline cellulose, croscarmellose sodium, lactose, magnesium stearate, hydroxypropyl cellulose, starch and talc. For example, the unit dose form may contain one or more of the following excipients: 5-15% microcrystalline cellulose, 0.5-5% croscarmellose sodium, 10-85% lactose, 0.5-5% magnesium stearate, 2-6% hydroxypropyl cellulose, 3-15% pregelatinized starch (e.g. Starch 1500), and/or 1-10% talc. In one embodiment the unit dose form comprises all of the all of the above excipients. It is most preferred, in this embodiment, that the tablet formulation comprises a therapeutically effective amount of ibuprofen or its pharmaceutically acceptable salts, in combination with famotidine with pharmaceutically acceptable excipients in a pharmacokinetically effective ratio. In one embodiment the excipients include microcrystalline cellulose 5-15% by weight, croscarmellose sodium 0.5-5% by weight, lactose 10-85% by weight, magnesium stearate 0.5-5% by weight, hydroxypropyl cellulose 2-6% by weight, pregelatinized starch 3-15% by weight and talc 1-10% by weight.

In the formulations of the invention, the excipients are present in an amount sufficient to allow for release of the ibuprofen and famotidine from the tablet after administration to a subject in need of this therapeutic combination in a fashion allowing for absorption into the blood at a time and concentration such that the therapeutic effects match that of ibuprofen administered alone and that of famotidine administered alone. As described in Example 3, it was demonstrated in human clinical studies that there are no significant differences between the pharmacokinetic parameters for either ibuprofen or famotidine when administered alone compared to administration in combination. It was concluded that both ibuprofen and famotidine can be considered bioequivalent when administered in combination compared to separate administration.

In a different embodiment, the pharmaceutical composition comprises microcrystalline cellulose 5-10% by weight, croscarmellose sodium 1-4% by weight, lactose 20-75% by weight, magnesium stearate 1-3% by weight, hydroxypropyl cellulose 3-5% by weight, pregelatinized starch 5-10% by weight and talc 2-6% by weight.

In another embodiment, the dosage for comprises 60-80% ibuprofen; 1.5-3.0% famotidine; 9-11% microcrystalline cellulose; 2-4% silicified microcrystalline cellulose; and 0.5-2.5% croscarmellose sodium.

Preferably the formulation comprises 60-80% ibuprofen; 1.5-3.0% famotidine; 9-11% microcrystalline cellulose; 2-4% silicified microcrystalline cellulose; 1-3% low substituted hydroxylpropylcellulose; and 0.5-2.5% croscarmellose sodium.

In one embodiment the formulation comprises ibuprofen, famotidine, microcrystalline cellulose, pregelatinized starch, hydroxypropyl cellulose, low substituted hydroxypropyl cellulose, silicon dioxide, silicified microcrystalline cellulose, croscarmellose sodium and magnesium stearate.

In one embodiment the formulation comprises 60-80% ibuprofen; 1.5-3.0% famotidine; 9-11% microcrystalline cellulose; 0.5-1.5% pregelatinized starch, 0.2-1% hydroxypropyl cellulose, 1-3% low substituted hydroxypropyl cellulose, 0.2-1% silicon dioxide, 2-4% silicified microcrystalline cellulose; 0.5-2.5% croscarmellose sodium, and 0.5-2.9% magnesium stearate.

In one embodiment the formulation comprises 76-78% ibuprofen; 1.5-2.5% famotidine; 9-11% microcrystalline cellulose; 0.5-1.5% pregelatinized starch, 0.2-1% hydroxypropyl cellulose, 1-3% low substituted hydroxypropyl cellulose, 0.2-1% silicon dioxide, 2-4% silicified microcrystalline cellulose; 0.5-2.5% croscarmellose sodium, and 0.5-2.9% magnesium stearate.

In certain embodiments the microcrystalline cellulose is comprised of a first population of particles having a median particle size of about 50 microns (e.g., EMOCEL 50M) and a second population of particles having a median particle size of approximately 90 microns (e.g., EMOCEL 90M). In some embodiments, 50-micron particles are present in at least 10-fold excess, and sometimes at least a 20-fold excess, over 90-micron particles.

In certain embodiments the silicified microcrystalline cellulose (SMCC) is comprised of a first population of particles having a median particle size of about 50 microns (e.g., PROSOLV 50 from Penwest) and a second population of particles having a median particle size of approximately 90 microns (e.g., PROSOLV 90 from Penwest). In one embodiment, the two populations are present in approximately equal quantities.

As shown in Example 8-4, inclusion of SMCC and low substituted hydroxypropylcellulose in the formulation resulted in tablets with better compressibility.

In one embodiment the unit dose form has the following composition:

| | |
|---|---|
| Famotidine | 1.5-2.5% |
| Microcrystalline cellulose (median particle size 50 microns) | 9-10% |
| Starch (pregelatinzed) | 0.8-10% |
| Hydroxypropyl cellulose | 0.4-0.8% |
| Ibuprofen | 70-80% |
| Colloidal silicon dioxide | 0.05-0.10% |
| Microcrystalline cellulose (median particle size 90 microns) | 0.2-0.6% |
| Silicified microcystalline cellulose (median particle size 50 | 1-2% |
| microns) | |
| Silicified microcrystalline cellulose (median particle size 90 | 1-2% |
| microns) | |
| Low substituted HPC | 1-2% |
| Croscarmellose sodium | 1-3% |
| Magnesium stearate | 2-2.9% |

In one embodiment the unit dose form has the following composition:

| | |
|---|---|
| Famotidine | 1.9% |
| Microcrystalline cellulose (median particle size 50 microns) | 9.6% |
| Starch (pregelatinzed) | 0.96% |
| Hydroxypropyl cellulose | 0.58% |
| Ibuprofen | 76.9% |
| Colloidal silicon dioxide | 0.08% |
| Microcrystalline cellulose (median particle size 90 microns) | 0.42% |
| Silicified microcystalline cellulose (median particle size 50 | 1.73% |
| microns) | |
| Silicified microcrystalline cellulose (median particle size 90 | 1.73% |
| microns) | |
| Low substituted HPC | 1.54% |
| Croscarmellose sodium | 2.0% |
| Magnesium stearate | 2.5% |

In one embodiment the unit dose form has the following composition:

| | | |
|---|---|---|
| Famotidine | 13.3 | mg |
| Microcrystalline cellulose (median particle size 50 microns) | 50.7 | mg |
| Pregelatinzed starch | 5 | mg |
| Hydroxypropyl cellulose | 3 | mg |
| Ibuprofen | 400.0 | mg |
| Colloidal silicon dioxide | 0.4 | mg |
| Microcrystalline cellulose (median particle size 90 microns) | 2.2 | mg |
| Silicified microcystalline cellulose (median particle size 50 | 9.0 | mg |
| microns) | | |
| Silicified microcrystalline cellulose (median particle size 90 | 9.0 | mg |
| microns) | | |
| Low substituted HPC | 8.0 | mg |
| Croscarmellose sodium | 10.4 | mg |
| Magnesium stearate | 13.0 | mg |
| total | 524.0 | mg |

In one embodiment the unit dose form has the following composition:

| Ingredient | % |
|---|---|
| Famotidine | 2.5 |
| Microcrystalline cellulose (e.g., Emcocel ® 50 M) | 9.7 |
| Pregelatinzed starch (e.g., Starch 1500) | 0.95 |
| Hydroxypropyl cellulose (e.g., Klucel EXF Pharm) | 0.57 |
| Ibuprofen 90 | 76.3 |
| Colloidal silicon dioxide | 0.08 |
| Microcrystalline cellulose (e.g., Emcocel ® 90M) | 0.42 |
| Silicified microcystalline cellulose (e.g., ProSolv | 1.72 |
| SMCC ® 50) | |
| Silicified microcrystalline cellulose (e.g., ProSolv | 1.72 |
| SMCC ® 90) | |
| Low substituted HPC (e.g., LH-11) | 1.53 |
| Croscarmellose sodium | 2.0 |
| Magnesium stearate | 2.5 |

### 11.0 ORAL DOSAGE FORMS CONTAINING FAMOTIDINE-NSAID FORMULATIONS

In another aspect, the invention is directed to a solid pharmaceutical composition for oral administration which comprises one or more non-steroidal anti-inflammatory (NSAID) compounds selected from the group consisting of aspirin, diclofenac, meclofenamate, mefenamic acid, meloxicam, nabumetone, naproxen, oxaprozin, phenylbutazone, piroxicam, sulindac, tenoxicam, diflunisal, tiaprofenic acid, tolmetin, etodolac, fenoprofen, floctafenine, flurbiprofen, indomethacin, and ketoprofen or a pharmaceutically acceptable salt thereof, in admixture with famotidine and one or more excipients, in a pharmacokinetically effective ratio such that said NSAID(s) and said famotidine are released in a bioequivalent manner.

In a particular embodiment, the NSAID and famotidine are released from said formulation simultaneously, at a rate and in a ratio providing each in a therapeutically effective and non-toxic amount. In another embodiment, the pharmaceutical composition is in a unit dose form. In yet another embodiment, the pharmaceutical composition is in the form of a tablet, pill, capsule, caplet, or gelcap.

In one embodiment, the compositions of the present invention do not contain any therapeutically active ingredient in addition to one or more NSAID and famotidine.

In a still further embodiment, the pharmaceutical composition comprises microcrystalline cellulose 5-15% by weight, croscarmellose sodium 0.5-5% by weight, lactose 10-85% by weight, magnesium stearate 0.5-5% by weight, hydroxypropyl cellulose 2-6% by weight, pregelatinized starch 3-15% by weight and talc 1-10% by weight.

In a different embodiment, the pharmaceutical composition comprises microcrystalline cellulose 5-10% by weight, croscarmellose sodium 1-4% by weight, lactose 20-75% by weight, magnesium stearate 1-3% by weight, hydroxypropyl cellulose 3-5% by weight, pregelatinized starch, 5-10% by weight and talc 2-6% by weight.

In other embodiments, the oral dosage forms containing famotidine in admixture with selected from the group consisting of aspirin, diclofenac, meclofenamate, mefenamic acid, meloxicam, nabumetone, naproxen, oxaprozin, phenylbutazone, piroxicam, sulindac, tenoxicam, diflunisal, tiaprofenic acid, tolmetin, etodolac, fenoprofen, floctafenine, flurbiprofen, indomethacin, and ketoprofen may be formulated as described herein for ibuprofen-famotidine forms.

### 12.0 METHOD OF MAKING TABLETS CONTAINING IBUPROFEN AND FAMOTIDINE

As described in the Examples, we have discovered that a tablet having suitable properties can be made using a wet granulation process and includes as components ibuprofen, famotidine, microcrystalline cellulose, silicified microcrystalline cellulose, and croscarmellose sodium.

In a related aspect, the invention provides methods for making ibuprofen/famotidine tablets with the above-described content and properties. In general it is desirable that tablets for oral administration have a high degree of uniformity as to weight and content, have dissolution properties appropriate for the API(s) being administered, and are chemically stable.

Methods for preparing tablets from a solid formulation are well known in the art. Briefly, tablets are usually formed by pressure applied to the material to be tabletted on a tablet press. A tablet press includes a lower punch which fits into a die from the bottom and an upper punch having a corresponding shape and dimension, which enters the die cavity from the top after the tabletting material fills the die cavity. The tablet is formed by pressure applied on the lower and upper punches. To prepare a tablet containing one or more active ingredients, the mixture to be compressed into the dosage forms should have certain physical characteristics for processing. Among other things, the mixture to be compressed must be free-flowing, must be lubricated, and must possess sufficient cohesiveness to ensure that the solid dosage form remains intact after compression. The ability of the material to flow freely into the die is important in order to provide for uniform filling of the die and continuous movement of the material from the source of the material, e.g. a feed hopper. The lubricity of the material is important in the preparation of the solid dosage forms in which the compressed material must be readily ejected from the punch faces.

Thus, compressibility and uniformity are important properties of a solid dosage formulation to be tabletted.

There are three general methods of preparation of materials to be included in a solid dosage form prior to compression: (1) direct compression; (2) dry granulation; and (3) wet granulation (including high shear mixer granulation and fluidized bed granulation).

In direct compression procedures, the materials to be included in the solid dosage form are compressed directly, without modifying the physical nature of the material itself. For solid dosage forms wherein the drug itself constitutes a substantial portion of the total weight of the solid dosage form, the use of direct compression is limited to those situations where the drug itself must exhibit physical characteristics, such as cohesiveness, that make it a good candidate for direct compression with the rest of the ingredients. Tablets containing famotidine as the sole active ingredient can be manufactured by direct compression. However, this approach is not ideal for manufacturing tablets comprising ibuprofen and famotidine, primarily due to the poor solubility and low cohesiveness of ibuprofen.

In dry granulation (also called “direct dry mixing”) procedures, the tablet components are mixed, followed by slugging, dry screening, lubricating, and compression into tablets. Dry granulation may be used where one of the constituents, either the drug or the diluent, has sufficient cohesive properties to be tabletted. A dry granulation approach to preparing ibuprofen/famotidine tablets is described in Example 8-1. Tablets made by this process exhibited poor content uniformity for famotidine (84-87%) and a poor dissolution rate for famotidine (92-95% famotidine released after 30 minutes in a dissolution test).

Wet granulation procedures includes mixing the powders to be incorporated into a solid dosage form in an appropriate blender (such as a twin shell blender or double-code blender), and then adding solutions of a binding agent to the mixed powders to obtained a granulation. Thereafter, the damp mass is screened (e.g. in a 6-, 8-, 15-, 25-mesh screen), and dried (e.g. by tray drying, using a fluid-bed dryer, a spray dryer, microwave, vacuum, or infra-red dryer). A wet granulation approach to preparing ibuprofen/famotidine tablets is described in Examples 3-5 and was shown to be superior. Wet granulation provided a pre-compression material with better wetting properties, easing disintegration and dissolution. In addition, the content uniformity of the tablets prepared was improved.

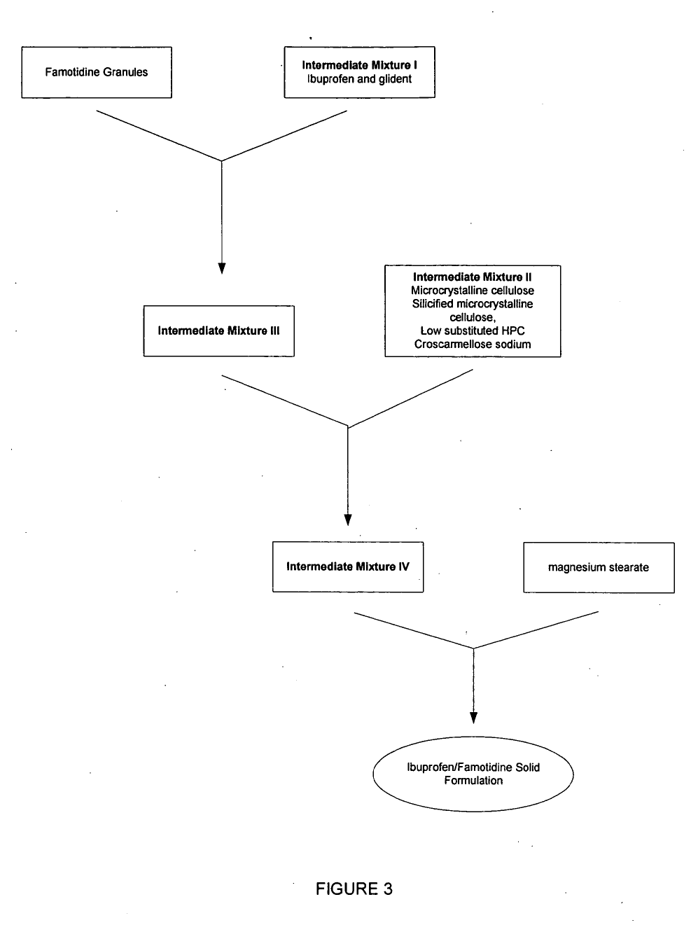

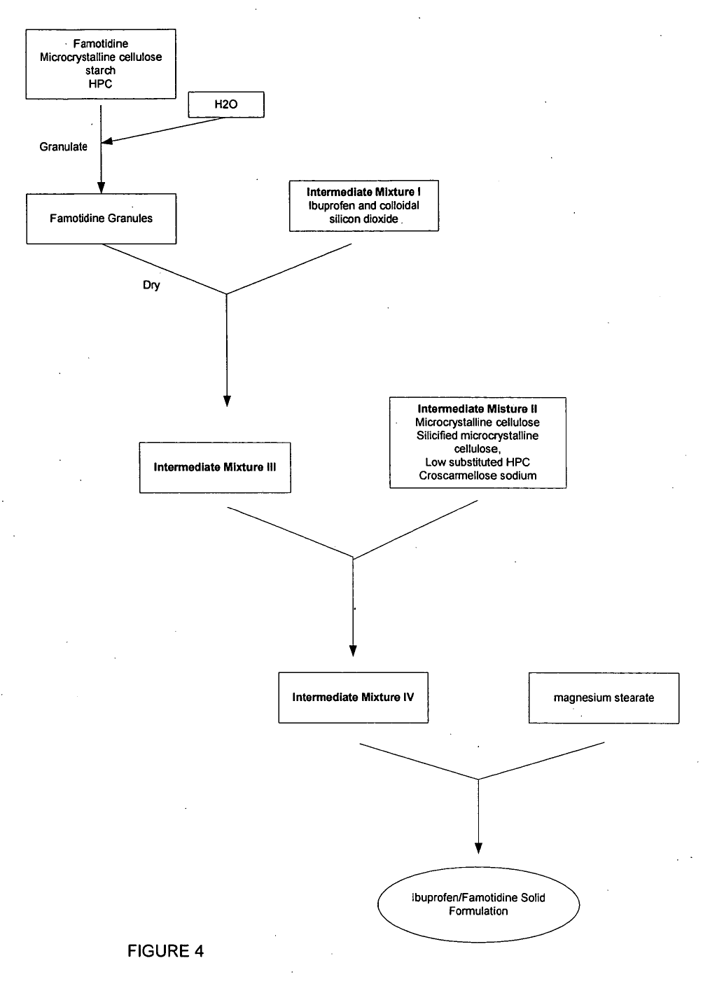

FIGS. 3 and 4 illustrate processes for making tablets containing the ibuprofen/famotidine compositions of the invention. In one aspect, the invention provides a method of making a tablet comprising ibuprofen and famotidine by:
a) preparing famotidine granules by wet granulation of 10 parts famotidine, 50 parts microcrystalline cellulose, 5 parts pregelatinized starch and 3 parts hydroxylpropyl cellulose, using water as the liquid, milling and screening the product;
b) mixing 400 parts ibuprofen and 0.4 parts colloidal silicon dioxide to produce intermediate mixture I;
c) mixing 2.2 parts microcrystalline cellulose, 9 parts SMCC 50, 9 parts SMCC90, 8 parts low substituted HPC, and 10.4 parts croscarmellose sodium to produce intermediate mixture II;
d) combining the intermediate mixture I and the famotidine granules incrementally by combining a first portion of intermediate mixture I with the famotidine granules and mixing, adding a second portion of intermediate mixture I and mixing, adding a third portion of intermediate mixture I and mixing, and optionally combining additional portions, thereby producing intermediate mixture III;
e) combining intermediate mixture II and intermediate mixture III to produce intermediate mixture IV;
f) adding 13 parts magnesium stearate to intermediate IV, thereby producing a ibuprofen/famotidine solid formulation; and,
g) compressing the ibuprofen/famotidine solid formulation to form tablets.

Using the methods described herein the solid pharmaceutical compositions of the invention can be formed into tablets with at least about 90%, at least about 95% or at least about 97% content uniformity.

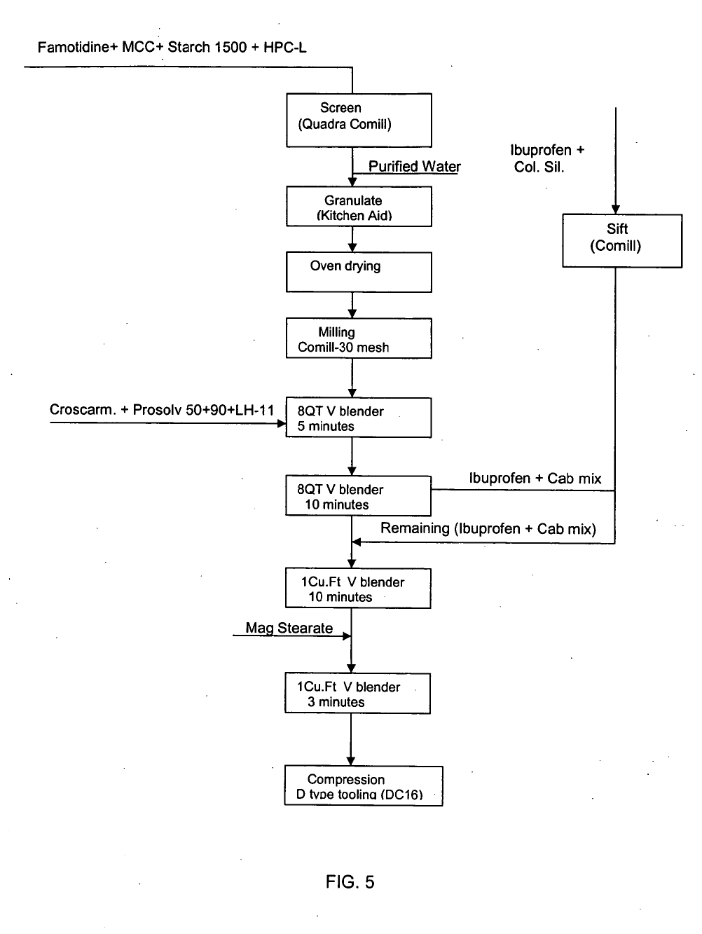

FIG. 5 illustrates a process for making tablets containing the ibuprofen/famotidine compositions of the invention. In one aspect, the invention provides a method of making a tablet comprising ibuprofen and famotidine by:
a) preparing famotidine granules by wet granulating famotidine in the presence of a binder and disintegrant and milling and screening the product;
b) mixing ibuprofen and a glident to produce an ibuprofen/glident mixture (intermediate mixture I);
c) mixing microcrystalline cellulose, silicified microcrystalline cellulose, low substituted HPC, and croscarmellose sodium (intermediate mixture II);
d) combining the famotidine granules with intermediate mixture II to produce intermediate mixture III;
e) combining intermediate mixture I and intermediate mixture III to produce intermediate mixture IV;
f) combining magnesium stearate to intermediate IV, thereby producing a ibuprofen/famotidine solid formulation; and,
g) compressing the ibuprofen/famotidine solid formulation to form tablets.

In one embodiment, the famotidine granules in (a) are prepared by combining and blending famotidine, microcrystalline cellulose, pregelatinized starch and hydroxypropyl cellulose, adding water as the granulating liquid, drying the famotidine, and milling and screening the product.

In one embodiment, the glident in step (b) is colloidal silicon dioxide.

In one embodiment the invention provides a method of making a tablet comprising ibuprofen and famotidine by:
a) preparing famotidine granules by wet granulation of 10 parts famotidine, 50 parts microcrystalline cellulose, 5 parts pregelatinized starch and 3 parts hydroxylpropyl cellulose, using water as the liquid, milling and screening the product;
b) mixing 400 parts ibuprofen and 0.4 parts colloidal silicon dioxide to produce intermediate mixture I;
c) mixing 2.2 parts microcrystalline cellulose, 9 parts SMCC 50, 9 parts SMCC90, 8 parts low substituted HPC, and 10.4 parts croscarmellose sodium to produce intermediate mixture H;
d) combining the intermediate mixture I and the famotidine granules incrementally by combining a first portion of intermediate mixture I with the famotidine granules and mixing, adding a second portion of intermediate mixture I and mixing, adding a third portion of intermediate mixture I and mixing, and optionally combining additional portions, thereby producing intermediate mixture III;
e) combining intermediate mixture H and intermediate mixture III to produce intermediate mixture IV;
f) adding 13 parts magnesium stearate to intermediate IV, thereby producing a ibuprofen/famotidine solid formulation; and,

g) compressing the ibuprofen/famotidine solid formulation to form tablets.

Using the methods described herein the solid pharmaceutical compositions of the invention can be formed into tablets with content uniformity (n=10) as shown below.

| | Mean (% Claim) | RSD |
|---|---|---|
| Ibuprofen | 102.3 | 2.6% |
| Famotidine | 101.4 | 1.9% |

Dissolution results indicated at least 95% of ibuprofen or famotidine released after 10 minutes (measured under neutral dissolution conditions).

### 13.0 PACKAGING

In one aspect the invention provides a container, such as a vial, containing a one-month supply of ibuprofen/famotidine tablets of the invention, wherein the number of tablets in the container is from 89-94 tablets (e.g., 89, 90, 91, 92, 93 or 94 tablets), and wherein instructions to take the medication 3× daily are affixed to the container, or packaged with the container.

Also provided is a container containing a two-month supply of ibuprofen/famotidine tablets of the invention, wherein the number of tablets in the container is 178-188 tablets, and wherein instructions to take the medication 3× daily are affixed to the container or packaged with the container.

### 14.0 METHOD OF TREATMENT

In another aspect, the invention provides a method of treating a patient in need of ibuprofen treatment, comprising prescribing or administering the ibuprofen/famotidine unit dose forms (tablets) of the invention. In one embodiment the patient is instructed to ingest the drug tablets three times daily. In one embodiment the patient is instructed to ensure there is at least a 6-hr interval between administrations of consecutive doses.

In one aspect the invention provides a method of treating a patient in need of ibuprofen treatment, where the patient is at elevated risk for developing an NSAID-induced ulcer. In one aspect the invention provides a method of treating a patient in need of ibuprofen treatment, where the patient is at high risk for developing an NSAID-induced ulcer.

In one aspect the invention provides a method of reducing, in a subject in need of ibuprofen treatment, the risk of developing an ibuprofen-induced symptom or condition such as, but not limited to, ulcer or GERD. This method involves administering to the subject an effective amount of a ibuprofen in admixture with an effective amount of famotidine, wherein the famotidine is administered three times per day. In an embodiment, the ibuprofen-induced condition is dyspepsia.

In a different aspect, the invention concerns a method of treating chronic pain, an inflammatory condition, or a condition associated with chronic pain or an inflammatory condition, comprising administering to a subject in need an effective amount of a pharmaceutical composition as hereinabove described.

The subject preferably is a human patient, and the condition to be treated may, for example, be selected from the group consisting of chronic pain, tenderness, inflammation, swelling, fever, headache, or stiffness caused by inflammatory conditions, muscle ache, menstrual pain, injuries, common cold, backache, and surgery or dental work related pain or inflammation. In a particular embodiment, the inflammatory condition is arthritis or gout.

In a still further aspect, the invention concerns a method for reducing the gastro-intestinal side-effects of a non-steroidal anti-inflammatory compound (NSAID), comprising administering said NSAID as part of a pharmaceutical composition comprising the non-steroidal anti-inflammatory (NSAID) compound, or a pharmaceutically acceptable salt thereof, and famotidine, in the absence of other non-NSAID therapeutically active ingredients, in admixture with one or more excipients, in a pharmacokinetically effective ratio such that said NSAID and said famotidine are released in a bioequivalent manner.

The present invention is also directed to a method of preventing the occurrence of gastrointestinal toxicity associated with the use of NSAIDs. In another embodiment, the present invention is directed to a method for preventing toxicities associated with NSAID use such toxicities include gastrointestinal ulceration, dyspepsia or upset stomach. In another embodiment, the present invention is directed to a method for preventing toxicities associated with NSAID use such toxicities include gastrointestinal ulceration, dyspepsia or upset stomach in patients who are specifically at risk for the development of such toxicities

### 15.0 MEDICAL USE

In a related aspect, the invention provides the use of famotidine in admixture with ibuprofen for the manufacture of a medicament for treatment of an ibuprofen responsive condition, wherein said medicament is adapted for oral administration in a unit dosage form for administration three times per day. In a preferred embodiment, the unit dosage form has an amount of famotidine such that TID administration delivers about 80 mg famotidine per day (e.g., about 13 mg or about 26 mg per unit dose form).

### 16.0 BUSINESS METHODS

Also provided is a business method comprising manufacturing, marketing, using, distributing, selling, or licensing, the ibuprofen-famotidine oral dosage forms of the invention. For example, the invention provides a method of doing business comprising (i) manufacturing ibuprofen/famotidine tablets of the invention, or having said tablets manufactured, and (ii) selling the ibuprofen/famotidine tablets to pharmacies or hospitals.

The invention also provides a method of doing business by advertising or selling a solid oral unit dosage form of the invention with instructions to take the dosage form on a TID schedule.

### 17.0 EXAMPLES

### 17.1 Example 1

### Administration of Famotidine-Ibuprofen TID Provides Protection Superior to that Provided by Administration of Famotidine QD and Ibuprofen TID.

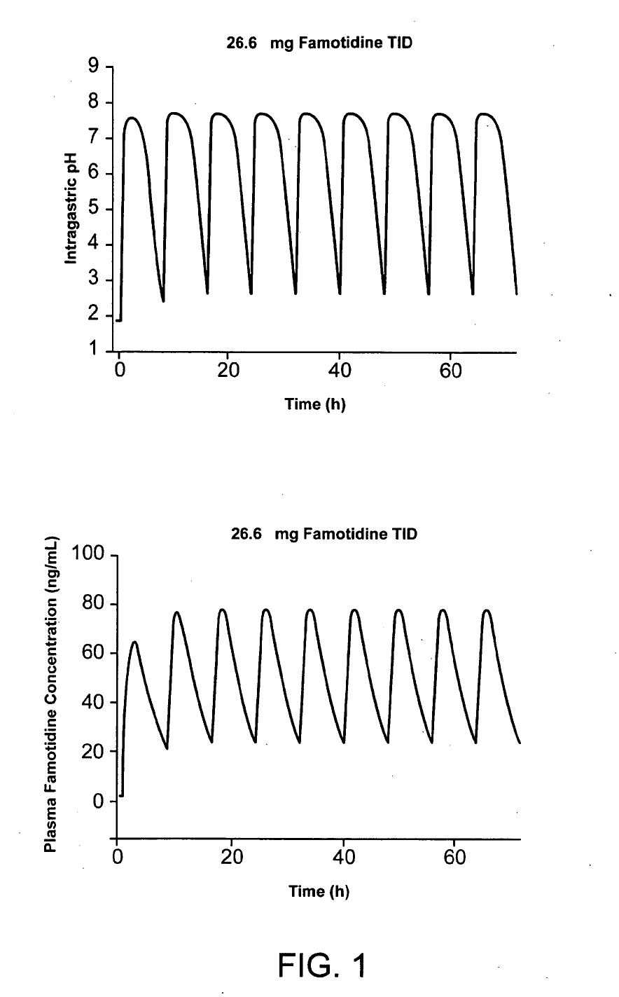

Pharmocokinetic modeling shows that TED administration of famotidine and ibuprofen according to the method of the present invention provides protection superior to that achieved by conventional cotherapy. FIG. 1A shows the predicted effect on intragastric pH of administration of 26.6 mg famotidine TID. FIG. 1B shows the predicted effect on intragastric pH of administration of 40 mg famotidine BID. Modeling shows that over a twenty-four hour interval, intragastric pH is greater than 3.5 during for several more hours per day than achieved using TID administration of famotidine compared to conventional BID dosing. In FIG. 1, administration of 80 mg/day famotidine using TID dosing is shown to maintain pH greater than 3.5 for about 21 hours per twenty-four hour interval, while the same daily dose administered BID dosing maintains pH greater than 3.5 for about 17 hours per twenty-four hour interval. The precise duration of pH elevation can be confirmed in clinical trials and may deviate somewhat from the predicted values (with the TID dosing remaining more effective than the BID dosing).

Methodology: Mean plasma concentration versus time data from a single dose bioequivalence study (www.fda.gov/cder/foi/anda/2001/75-311_Farnotidine_Bioeqr.pdf, n=30) comparing 40 mg Pepcid and generic famotidine (Teva Pharm) were best fitted to a one compartment oral absorption model with a lag time using a nonlinear least-squares regression program, WinNonlin (Pharsight®). The following pharmacokinetic parameters for Pepcid were obtained:

| Parameter | Units | Estimate |
|---|---|---|
| V/F | L | 241.8 |
| kₐ | h⁻¹ | 0.8133 |
| kₑₗ | h⁻¹ | 0.2643 |
| T_{lag} | h | 0.3677 |

where V/F is the apparent volume of distribution, kₐ is the absorption rate constant, kₑₗ is the elimination rate constant and T_{lag} is the absorption lag time.

The relationship between plasma concentrations of Pepcid and intragastric pH in one patient were digitized from FIG. 4 of Echizen and Ishizaki, supra, page 189. The digitized plasma concentration vs. intragastric pH were fitted using a nonlinear least-squares regression program, WinNonlin to a sigmoid Emax model using the following equation:

$$E = {E}_{o} + \frac{{E}_{max} * {C}^{γ}}{{EC}_{50}^{γ} * {C}^{γ}}$$

where E is the intragastric pH at C, Eₒ is the intragastric pH at time zero, Eₘₐₓ is the maximum intragastric pH, EC₅₀ is the Pepcid concentration at one-half of Emax, C is the plasma concentration of Pepcid and γ is the shape factor. The estimated pharmacodynamic parameters are listed below:

| Parameter | Units | Estimate |
|---|---|---|
| Eₘₐₓ | — | 7.80 |
| EC₅₀ | ng/mL | 32.6 |
| E₀ | — | 1.88 |
| γ | — | 4.80 |

Using the pharmacokinetic parameters obtained above together with the pharmacodynamic parameters above, plasma concentrations as well as intragastric pH as a function of time were simulated for various dose regimens.

### 17.2 Example 2

### Administration of Famotidine TID Provides Superior Gastric Protection Compared to Administration of Famotidine QD

A randomized, open-label, two-period, crossover study is carried out to compare the effects on gastric pH of administration of 80 mg per day of famotidine when administered for five consecutive days in two versus three divided doses each day.

Healthy male or female subjects, age 18-45 years inclusive, are randomized to treatment to ensure that at least 12 subjects will complete study participation. Subjects are assigned randomly, in approximately a 1:1 ratio, to one of two, two-period treatment sequences as follows:
Treatment Sequence 1: 40 mg famotidine BID×5 days, followed by 26.6 mg famotidine TID×5 days.
Treatment Sequence 2: 26.6 mg famotidine TID×5 days, followed by 40 mg famotidine BID×5 days.

There is a washout of at least one week between administration of the last dose of Treatment Period 1 and administration of the first dose of Treatment Period 2.

PEPCID® (famotidine) for Oral Suspension (Merck & Co., Inc., 40 mg/5 mL) is administered with water. During treatment periods in which famotidine is to be administered TID, medication is administered at approximately 0800, 1600, and 2400 on each day of dosing. During treatment periods in which famotidine is to be administered BID, medication is administered at approximately 0800 and 2000 on each day of dosing.

Gastric pH is measured continuously, using a nasogastric pH probe, during the 24 hours following administration of the first dose of study medication on Study Day 1, and during the 24 hours following administration of the first dose of study medication on Study Day 5, during both treatment periods. Blood samples are collected prior to initiation of dosing, and prior to administration of the second dose of study medication on Study Day 1 and Study Day 5 during both treatment periods for determination of trough plasma famotidine concentrations.

The effect of each dose regimen, and the difference between the two dosing regimens, is estimated by the 95% confidence intervals for the variables (i) mean and median pH during the final 24-hour measurement period of each treatment period, and (ii) percentage of time during the final 24-hour measurement period of each treatment period in which the pH is below 4, when 80 mg doses of famotidine are administered for five consecutive days in two versus three divided doses each day. An analysis of variance (ANOVA) will be performed to estimate the effects of each dose regimen and to compare the two dosing regimens for both efficacy variables.

It is expected that administration of famotidine TID provides superior protection, as measured by gastric pH, compared to therapy with famotidine BID. TID administration of famotidine maintains a gastric pH greater than pH 3.0 more than 1 hour longer per 24-hour dosing cycle than does BID administration. TID administration of famotidine results in a minimum sustained pH that is at least 0.2 pH units higher than BID administration. TID administration of famotidine results in an average gastric pH that is at least 0.2 pH units higher than BID administration.

### 17.3 Example 3

### Pharmacokinetic Drug-Drug Interaction Study of Ibuprofen and Famotidine in Healthy Male Subjects

This example demonstrates that pharmocokinetic parameters of concurrent administration of ibuprofen and famotidine (as in the unit dose forms of the invention) are bioequivalent to separate administration of the two APIs. An open-label, randomized, single-dose, oral administration, two-period crossover study was conducted. Six male subjects were assigned randomly to Sequence 1 or Sequence 2:

Sequence 1
Period 1: 800 mg ibuprofen [Motrin®], followed 24 hr later by 40 mg famotidine [Pepcid®].
Period 2: Concurrent administration of 800 mg ibuprofen and 40 mg famotidine.

Sequence 2
Period 1: Concurrent administration of 800 mg ibuprofen and 40 mg famotidine.
Period 2: 800 mg of ibuprofen, followed 24 hr later by 40 mg famotidine.

Following administration of ibuprofen and famotidine plasma ibuprofen and/or famotidine concentrations were determined in samples collected predose and at 0.25, 0.5, 1.0, 1.5, 2, 4, 6, 8, 10, 12, 14, 18, and 24 hr after administration of ibuprofen and/or famotidine. Ibuprofen and famotidine plasma concentrations, and computed pharmacokinetic parameters, were listed and summarized by dose (mean, standard deviation, 95% confidence interval, minimum, maximum). Individual and mean (by time) concentration-versus-time curves for each treatment, plotted on a semi-log scale, were examined. Intra-subject comparisons were made between Period 1 and Period 2.

WinNonLin version 2.1 was used to analyze the pharmacokinetic parameters from the concentration-versus-time data based a non-compartmental model. The pharmacokinetic values then were transferred to MS Excel or Graphpad Prism for calculation of means, SDs, confidence intervals, etc., for preparation of tables and figures, and for performance of statistical testing.

Analyses of variance appropriate for a two-period crossover design were performed on the computed parameters including terms for sequence, subject within sequence, formulation; and period. Analyses were performed on the observed data and on natural logarithm-transformed data for area under the concentration-versus-time curve (AUC) and maximum observed plasma concentration (Cₘₐₓ). Ninety-five (95) % confidence intervals were computed for the differences in treatment means.

After confirming the absence of a period effect for the pharmacokinetic parameters, individual AUC and Cₘₐₓ data were pooled for each treatment (i.e., for both ibuprofen and famotidine administered alone and in combination) for bioequivalence testing. The individual data then were log-transformed (natural log) and the differences for each drug between administration alone versus in combination were determined for each subject. The means and 95% confidence intervals of these log-transformed differences were calculated, and the upper and lower bound of the log-transformed range were normalized and then tested for bioequivalence. These intervals were evaluated in relation to the criterion equivalence interval of 80% to 125% for log-transformed data. Tables 1-3 show the results of the analyses:

TABLE 1
Pharmacokinetic Parameters (mean ± SD, 95% CI) for Ibuprofen and
Famotidine When Administered Alone and In Combination

| | Ibuprofen | | Famotidine | |
|---|---|---|---|---|
| Parameter | Alone | With Famotidine | Alone | With Ibuprofen |
| tₘₐₓ (hr) | 1.58 ± 0.49 | 2.25 ± 1.89 | 1.67 ± 0.52 | 2.17 ± 0.93 |
| (95% CI) | (1.07-2.10) | (0.27-4.23) | (1.13-2.21) | (1.19-3.14) |
| Cₘₐₓ | 56,279 ± 8,486 | 55,666 ± 12,106 | 143 ± 31 | 159 ± 50 |
| (ng/mL) | (47,374-65,184) | (42,961-68,370) | (111-175) | (107-211) |
| (95% CI) | | | | |
| t_{1/2} (hr) | 2.50 ± 0.55 | 2.56 ± 0.59 | 3.66 ± 0.19 | 3.49 ± 0.35 |
| (95% CI) | (1.92-3.07) | (1.95-3.18) | (3.46-3.86) | (3.12-3.85) |
| Kₑₗ | 0.29 ± 0.06 | 0.28 ± 0.06 | 0.19 ± 0.01 | 0.20 ± 0.02 |
| (95% CI) | (0.23-0.35) | (0.22-0.34) | (0.18-0.20) | (0.18-0.22) |
| AUC₍ₗₐₛₜ₎ | 236,992 ± 62,862 | 234,851 ± 67,655 | 883 ± 173 | 934 ± 275 |
| (ng/mL · hr) | (171,023-302,961) | (163,851-305,850) | (701-1064) | (646-1222) |
| (95% CI) | | | | |
| AUC | 245,124 ± 63,697 | 235,156 ± 67,749 | 893 ± 175 | 944 ± 279 |
| (ng/mL · hr) | (178,279-311,970) | (164,058-306,254) | (710-1077) | (651-1236) |
| (95% CI) | | | | |

TABLE 2
Bioequivalence Test Results for AUC (log-transformed values)
for Ibuprofen and Famotidine When Administered
Alone Versus In Combination

| | | AUC₍ₗₐₛₜ₎ | | |
|---|---|---|---|---|
| Drug | AUC₍ₗₐₛₜ₎ Alone | In Combination | Difference | 95% CI |
| Ibuprofen | 12.35 | 12.33 | 0.02 | 0.94-1.11 |
| Famotidine | 6.765 | 6.799 | −0.034 | 0.79-1.19 |

¹Test criterion: CI within 0.8-1.25

TABLE 3
Bioequivalence Test Results for Cₘₐₓ (log-transformed values)
for Ibuprofen and Famotidine When Administered
Alone Versus In Combination

| | Cₘₐₓ | | | |
|---|---|---|---|---|
| Drug | Alone | Cₘₐₓ In Combination | Difference | 95% CI |
| Ibuprofen | 10.93 | 10.91 | 0.02 | 0.85-1.23 |
| Famotidine | 4.94 | 5.02 | −0.08 | 0.76-1.12 |

¹Test criterion: CI within 0.8-1.25

There were no significant differences between the treatment means for the pharmacokinetic parameters for either ibuprofen or famotidine when administered alone versus in combination. It was concluded that both ibuprofen and famotidine can be considered bioequivalent when administered in combination compared to separate administration.

### 17.4 Example 4

### Ibuprofen-Famotidine Compatibility Studies

As shown in Table 4, substantial degradation of famotidine was observed in the famotidine-ibuprofen mixture (1:29 ratio) under stress conditions in the presence of ibuprofen. In the absence of ibuprofen, famotidine is stable.

TABLE 4
Famotidine/Ibuprofen Stability Under Stress Conditions

| | | Famotidine |
|---|---|---|
| API | Storage condition | Content* |
| Famotidine | 2 weeks at 60° C. | 98% |
| Famotidine + Ibuprofen | 2 weeks at 60° C. | 81% |
| Famotidine + Ibuprofen | 1 mo at 40° C./75% RH | 54% |

*Famotidine content was determined by analytical HPLC and expressed as percent of target content.

Similarly, as shown in Table 5 substantial degradation of famotidine was observed in the tablet dosage form containing ibuprofen in the tablet formulation under stress conditions.

TABLE 5
Stability of Famotidine in Tablet Under Stress Conditions

| Drugs in Tablet | | Famotidine |
|---|---|---|
| Formulation | Storage condition | Content* |
| Famotidine (13.3 mg) + | Initial | 100% |
| Ibuprofen (400 mg) | | |
| Famotidine (13.3 mg) + | 1 week at 60° C. | 39% |
| Ibuprofen (400 mg) | | |
| Famotidine (13.3 mg) + | 1 month at | 83% |
| Ibuprofen (400 mg) | 40° C./75% RH | |

*Famotidine content was determined by analytical HPLC and expressed as percent of target content.

Similarly, as shown in Table 6 substantial degradation of famotidine was observed in the tablet dosage form containing ibuprofen in the tablet formulation under stress conditions. However, the famotidine is stable when stored at room temperature in the tablet form.

TABLE 6
Famotidine/Ibuprofen Stability Under Stress Conditions

| | | Amt. of |
|---|---|---|
| API | Storage condition | famotidine |
| Famotidine (10 mg) + | 4 months, room | 99% |
| Ibuprofen (400 mg) | temperature | |
| in tablet form with | | |
| excipients | | |
| Famotidine (10 mg) + | 1 month at 60° C | 4% |
| Ibuprofen (400 mg) | | |
| in tablet form with | | |
| excipients | | |

“Amt. of famotidine” refers to the amount of famotidine remaining at the end of the storage period (as % of original content). Famotidine content was determined by analytical HPLC.

### 17.5 Example 5

### Additional Ibuprofen-Famotidine Compatibility Studies

Approximately 0.5 g famotidine API was mixed with 14.5 g ibuprofen. After grinding, API mixture was stored in glass vials under the conditions indicated. As shown in Table 7, substantial degradation of famotidine was observed.

TABLE 7
Famotidine/Ibuprofen Stability Under Stress Conditions

| | Ibuprofen (% control) | | | Famotidine (% control) | | |
|---|---|---|---|---|---|---|
| API | 1 wk | 1 wk | 2 wks | 1 wk | 1 wk | 2 wks |
| Mixture | 40° C. | 60° C. | 60° C. | 40° C. | 60° C. | 60° C. |
| Famotidine | | | | 96.1 | 121.0 | 100.1 |
| Famotidine- | 104.7 | 99.9 | 96.4 | 94.4 | 85.7 | 46.0 |
| Ibuprofen | | | | | | |

### 17.6 Example 6

### Determination of Dissolution

One method for determination of the rate and extent of dissolution can be carried out using the methods described in the United States Pharmacopeia and National Formulary 29th Revision, under the following conditions:
Dissolution Apparatus: Apparatus II (Paddles)
Dissolution Medium: 50.0 mM Potassium Phosphate Buffer, pH 7.2
Dissolution Medium Volume: 900 mL
Temperature in Vessel: 37.0° C.±0.5° C.
Speed: 50 RPM
Sampling Time: 10 mM., 20 min., 30 mM., 45 mM., 60 min., and infinity @ 250 rpm for 15 mM.
Sampling Volume: 1 mL
Sinker: None
   When desired, the dissolution medium or other parameters may be varied. Typically a unit dose form is added to the vessel and dissolution is started. At specified times a portion (e.g., 2 ml) of medium is withdrawn and the amount of API in solution is determined using routine analytical methods (e.g., HPLC).

### 17.7 Example 7

### Dissolution Properties of Tablets

Tablets containing ibuprofen (400 mg) and famotidine (10 mg) were prepared as described above in Example 8.3. Dissolution was determined essentially as described in Example 6. Dissolution properties are shown in Table 8 (phosphate buffer, pH 7.2) and Table 9 (phosphate buffer, pH 4.5).

TABLE 8
Dissolution Properties at Neutral pH

| Time | Ibuprofen | | Famotidine | |
|---|---|---|---|---|
| Point | (Buffer | | (Buffer pH | |
| (min) | pH7.2) | % RSD | 7.2) | % RSD |
| 10 | 95.4 | 5.4 | 79.8 | 2.6 |
| 20 | 96.5 | 4.5 | 83.9 | 1.8 |
| 30 | 96.7 | 4.1 | 85.5 | 1.1 |
| 45 | 97.4 | 3.2 | 87.3 | 0.9 |
| 60 | 97.5 | 3.1 | 88.2 | 0.9 |
| Inf. 1* | 99.1 | 1.0 | 90.7 | 2.5 |
| Inf. 2** | 101.6 | 1.1 | 94.4 | 2.4 |

*Inf. 1: 15 min@ 250 rpm
**Inf. 2: overnight@ 250 rpm

TABLE 9
Dissolution Properties at Low pH

| Time | Ibuprofen | | Famotidine | |
|---|---|---|---|---|
| Point | (Buffer | | (Buffer pH | |
| (min) | pH4.5) | % RSD | 4.5) | % RSD |
| 10 | 13.6 | 2.6 | 88.9 | 2.3 |
| 20 | 19.2 | 1.4 | 91.3 | 1.3 |
| 30 | 22.4 | 1.2 | 92.0 | 0.7 |
| 45 | 24.4 | 1.1 | 92.7 | 0.8 |
| 60 | 24.9 | 0.5 | 93.0 | 1.2 |
| Inf. 1* | 25.2 | 0.3 | 93.0 | 1.3 |
| Inf. 2** | 25.9 | 0.2 | 96.4 | 1.5 |

*Inf. 1: 15 min@250 rpm
**Inf. 2: overnight@250 rpm

### 17.8 Example 8

### Manufacture of Ibuprofen/Famotidine Unit Dose Forms

### Example 8-1

### Preparation of Ibuprofen/Famotidine Formulations by Direct Blending

TABLE 10

| Item # | Ingredient | % in formulation | mg/unit |
|---|---|---|---|
| 1 | Ibuprofen 90 | 83.23 | 400.0 |
| 2 | Famotidine | 2.08 | 10.0 |
| 3 | Colloidal silicon dioxide | 0.29 | 1.38 |
| 4 | Microcrystalline cellulose | 10.45 | 50.22 |
| | (EMCOCEL ® 90M) | | |
| 5 | Croscarmellose sodium | 1.91 | 9.20 |
| | (VivaSol ®) | | |
| 6 | Magnesium stearate | 2.00 | 9.60 |

| | | |
|---|---|---|
| Theoretical weight: | 480.4 | mg |

The ingredients listed in Table 10 were used to prepare an ibuprofen/famotidine formulation by dry mixing, following the steps listed below.

(1) Item #1 (ibuprofen) was passed through a 25-mesh screen into a polyethylene bag.

(2) Item #3 (colloidal silicon dioxide) was added to the polyethylene bag, which was then manually shaken 30-times.

(3) The materials from step (2) were then passed through a 25-mesh screen into another polyethylene bag and manually shaken 30-times.

(4) Item #2 (famotidine) was passed through a 25-mesh screen into a polyethylene bag.

(5) Item #4 (microcrystalline cellulose) was de-lumped through a 25-mesh screen into a polyethylene bag.

(6) 10 g of the de-lumped microcrystalline cellulose from step (5) was transferred into the step (4) screened famotidine bag, and the mixture was shaken 30-times.

(7) 10 g of the de-lumped microcrystalline cellulose from step (5) was transferred into the bag of step (6), which was shaken 30-times.

(8) All remaining de-lumped microcrystalline cellulose from step (5) was added into the bag of step (7), followed by shaking 30-times.

(9) The blend of step (8) was passed through a 25-mesh screen and mixed 30-times.

(10) 60 g of the blend of step (3) was transferred into the bag of step (9), followed by mixing 30-times, and massing through a 25-mesh screen.

(11) 120 g of the blend of step (3) was transferred into the bag of step (10), mixed 30-times, and passed through a 25-mesh screen.

(12) Item #5 (croscarmellose sodium) was de-lumped through a 25-mesh screen into the blend of step (11).

(13) All of the step (3) blend was transferred into the bag of step (12) and mixed 30-times.

(14) Item #6 (magnesium stearate) is passed through a 35-mesh screened into a polyethylene bag. An equal amount of the blend from step (13) is added to the bag, and is manually shaken 25-times. The mix is then added into the bag of step (13), and the mixture obtained is manually shaken 30-times.

(15) The mixture was compressed into tablets, using a Manesty D3B rotary tablet press.

The average weight of the tablets obtained was 480.6 (range 456.6-504.6 mg). The tablets made by this process exhibited poor content uniformity for famotidine content (84-87%) and a poor dissolution rate for famotidine (92-95% famotidine released after 30 minutes as measured using the USP dissolution test).

### Example 8-2

### Preparation of Ibuprofen/Famotidine Formulations Using Wet Granulation

The following ingredients were processed by a manufacturing procedure, which involved wet granulation, oven drying and Comil milling, as described below.

TABLE 11

| Item | | % in | |
|---|---|---|---|
| # | Ingredient | formulation | mg/unit |
| 1 | Famotidine | 1.9 | 10 |
| 2 | Microcrystalline cellulose | 9.6 | 50 |
| | (Emcocel ® 50 M) | | |
| 3 | Starch 1500 | 0.96 | 5 |
| 4 | Hydroxypropyl cellulose | 0.58 | 3 |
| | (Klucel EXF) | | |
| 5 | Purified water | | (removed) |
| | Sub-total 1 | | 68 |
| 6 | Ibuprofen 90 | 76.9 | 400.0 |
| 7 | Colloidal silicon dioxide | 0.29 | 1.5 |
| 8 | Microcrystalline cellulose | 4.71 | 24.5 |
| | (Emcocel ® 90M) | | |
| 9 | Croscarmellose sodium (VivaSol ®) | 2.0 | 10.4 |
| 10 | Magnesium stearate | 3.0 | 15.6 |
| | Sub-total 2 | | 452.0 |

| | | |
|---|---|---|
| Total | 520.0 | mg |

The tablets were prepared by the following procedure:

(1) Items 1-4 (famotidine, microcrystalline cellulose, starch 1500, hydroxypropyl cellulose) were passed through a 25-mesh screen into a polyethylene bag. The ingredients were then placed into a V-Blender and mixed for 5 minutes.

(2) The blend from step (1) was transferred into a low shear granulator (Kitchen Aid).

(3) The granulator was turned on at low speed, and item 5 (purified water) was added slowly into the blend until finish.

(4) The granulator was stopped and the wet granules were checked. Additional water was added until the wet granulation reached the end point. The total amount of purified water added was 265 ml.

(5) The oven is set at 50° C.

(6) The oven drying tray was covered with aluminum foil and the wet granules were evenly spread on the aluminum foil and dried at 50° C. Drying was stopped until water content is less than 3%.

(7) Dried granules were passed through a Comil equipped with a 032R screen into a polyethylene bag. At the end of milling, the residues were ground in a mortar and pestle, then passed through a 032R screen by hand.

(8) Item 6 (ibuprofen 90) was passed through a 25-mesh screen into a polyethylene bag.

(9) Item 7 (colloidal silicon dioxide) was added into the step (1) bag, and manually shaken 30-times.

(10) The step (9) materials were passed through a 25-mesh screen into a V-blender and mixed for 20 minutes.

(11) 68 g of the milled granules from step (7) were weighed and placed into a polyethylene bag.

(12) Approximately 60 g of the blend from step (10) was transferred into the bag of step (11), and the mixture was mixed in a V-blender for 5 minutes.

(13) Approximately 110 g of the blend from step (10) was transferred into the blend of step (12), and mixed in a V-blender for 5 minutes.

(14) The rest of the step (10) blend was transferred into the blend of step (13), and mixed in a V-blender for 5 minutes. The blend was then collected in a polyethylene bag.

(15) Items 8 and 9 (microcrystalline cellulose; croscarmellose sodium) were de-lumped by passing through a 25-mesh screen into a polyethylene bag. The mixture was manually shaken 30-times.

The tablets prepared using the method above had improved characteristics in terms of content uniformity and dissolution (nearly 100% after 30 minutes).

### Example 8-3

### Preparation of Ibuprofen/Famotidine Formulations Using Wet Granulation

Using a procedure similar to that described in Example 8-2, tablets with the following composition were prepared:

TABLE 12

| | | % in | |
|---|---|---|---|
| Item # | Ingredient | formula | mg/unit |
| 1 | Famotidine | 1.9 | 10 |
| 2 | Microcrystalline cellulose | 9.6 | 50 |
| | (Emcocel ® 50 M) | | |
| 3 | Pregelatinized starch (Starch 1500) | 0.96 | 5 |
| 4 | Hydroxypropyl cellulose (Klucel EXF) | 0.58 | 3 |
| 5 | Purified water | | (removed) |
| | Sub-total 1 | | 68 |
| 6 | Ibuprofen 90 | 76.9 | 400.0 |
| 7 | Colloidal silicon dioxide | 0.08 | 0.4 |
| 8 | Microcrystalline cellulose | 0.42 | 2.2 |
| | (Emcocel ® 90M) | | |
| 9 | SMCC (ProSolv ® 50) | 1.73 | 9.0 |
| 10 | SMCC (ProSolv ® 90) | 1.73 | 9.0 |
| 11 | Low substituted HPC (LH-11) | 1.54 | 8.0 |
| 12 | Croscarmellose sodium (VivaSol ®) | 2.0 | 10.4 |
| 13 | Magnesium stearate | 2.5 | 13.0 |
| | Sub-total 2 | | 452.0 |

| | |
|---|---|
| Total weight | 520.0 |

The composition of this formulation differs from the formulation of Example 3 in the addition of two type of silicified microcrystalline cellulose and low substituted HPC, and lowering the amount of magnesium stearate.

Compressibility: Tablets made with this formulation had significantly improved compressibility.

Uniformity: Both average and individual contents of the tablets met the USP specification of 100±15%. When subjected to uniformity testing individually weighed tablets had an average content of 95.58% with a relative standard deviation (RSD) of 6.2% which does not meet USP specifications of not more than 6%.

### Example 8-4

### Preparation of Ibuprofen/Famotidine Formulations Using Wet Granulation

To achieve better content uniformity, the procedure described in Example 8-3 was modified to improve mixing efficiency. Following steps 1-7 described in Example 8-2, the final blending stage of the manufacturing process was conducted as follows:

(1) Items 1-4 (famotidine, microcrystalline cellulose, starch 1500, hydroxypropyl cellulose) were passed through a 25-mesh screen into a polyethylene bag. The ingredients were then placed into a V-Blender and mixed for 5 minutes.

(2) The blend from step (1) was transferred into a low shear granulator (Kitchen Aid).

(3) The granulator was turned on at low speed, and Item 5 (purified water) was added slowly into the blend until finish.

(4) The granulator was stopped and the wet granules were checked. Additional water was added until the wet granulation reached the end point. The total amount of purified water added was 265 ml.

(5) The oven is set at 50° C.

(6) The oven drying tray was covered with aluminum foil and the wet granules were evenly spread on the aluminum foil and dried at 50° C. Drying was stopped until water content is less than 3%.

(7) Dried granules were passed through a Comil equipped with a 032R screen into a polyethylene bag. At the end of milling, the residues were ground in a mortar and pestle, then passed through a 032R screen by hand.

(8) Item 7 (colloidal silicon dioxide) was passed through a 25-mesh screen into a polyethylene bag.

(9) Item 8 (microcrystalline cellulose) was added into the bag of step (1) and was manually shaken 30 times.

(10) Step (9) materials were passed through a 25-mesh screen into a polyethylene bag and transferred into a 2 qt. V-blender. The contents were mixed for 30 minutes.

(11) 68 g of the famotidine granules were weighed and placed into a polyethylene bag.

(12) Approximately 60 g of the step (10) blend and step (11) granules were transferred into the blender of step (12) and mixed for 5 minutes.

(13) Approximately 110 g of the Step (10) blend were transferred into the V-blender of step (12) and mixed for 5 minutes.

(14) The rest of the Step (10) blend was transferred into the V-blender of step (13) and mixed for 5 minutes.

(15) Items 9-12 (Emcocel® 90M, ProSolv SMCC 50, ProSolv SMCC 90, LH-11, VivaSol) were de-lumped by passing through a 25-mesh screen into a polyethylene bag and manually shaken 30-times.

(16) 35 g of the step (14) blend were transferred into the bag of step (15) and manually shaken 30-times.

(17) 60 g of the step (14) blend were transferred into the bag of step (16) and manually shaken 30-times.

(18) 120 g of the step (14) blend and the step (17) blend were transferred into a 2 qt V-blender, and mixed for 5 minutes.

(19) The rest of step (14) blend was transferred into the step (18) V-blender, followed by mixing for 5 minutes.

(20) Item 13 (magnesium stearate) was passed through a 35-mesh screen into a polyethylene bag. An equal amount (13 g) of the blend from step (19) was added to the beg and manually shaken 25-times. Another equal amount (26 g) of the blend from step (19) was added to the bag and manually shaken 25-times. Then, it was added into the blender of step (19), followed by mixing for 5 minutes.

### Example 8-5

### Preparation of Ibuprofen/Famotidine Formulations Using Wet Granulation

To achieve better content uniformity, the procedure described in Example 8-4 was modified to add the Intermediate Mixture II prior to mixing with Intermediate Mixture I (ibuprofen and colloidal silicon dioxide). The famotidine content was increased to 13.3 mg/tablet. The process is summarized in FIG. 5.

TABLE 13

| | | % in | |
|---|---|---|---|
| Item # | Ingredient | formula | mg/unit |
| 1 | Famotidine | 2.5 | 13.3 |
| 2 | Microcrystalline cellulose | 9.68 | 50.7 |
| | (Emcocel ® 50 M) | | |
| 3 | Pregelatinized starch (Starch 1500) | 0.95 | 5 |
| 4 | Hydroxypropyl cellulose (Klucel EXF) | 0.57 | 3 |
| 5 | Purified water | | (removed) |
| 6 | Ibuprofen 90 (BASF) | 76.34 | 400.0 |
| 7 | Colloidal silicon dioxide | 0.08 | 0.4 |
| 8 | Microcrystalline cellulose | 0.42 | 2.2 |
| | (Emcocel ® 90M) | | |
| 9 | SMCC (ProSolv ® 50) | 1.72 | 9.0 |
| 10 | SMCC (ProSolv ® 90) | 1.72 | 9.0 |
| 11 | Low substituted HPC (LH-11) | 1.53 | 8.0 |
| 12 | Croscarmellose sodium (VivaSol) | 1.98 | 10.4 |
| 13 | Magnesium stearate | 2.48 | 13.0 |

| | |
|---|---|
| Total weight | 524.0 |

(1) Items 1-4 (famotidine, microcrystalline cellulose, starch 1500, hydroxypropyl cellulose) were passed through a 25-mesh screen into a polyethylene bag. The ingredients were then placed into a V-Blender and mixed for 5 minutes.

(2) The blend from step (1) was transferred into a low shear granulator (Kitchen Aid).

(3) The granulator was turned on at low speed, and Item 5 (purified water) was added slowly into the blend until finish.

(4) The granulator was stopped and the wet granules were checked. Additional water was added until the wet granulation reached the end point. The total amount of purified water added was 265 ml.

(5) The oven is set at 50° C.

(6) The oven drying tray was covered with aluminum foil and the wet granules were evenly spread on the aluminum foil and dried at 50° C. Drying was stopped until water content is less than 3%.

(7) Dried granules were passed through a Comil equipped with a 30 mesh screen into a polyethylene bag. At the end of milling, the residues were ground in a mortar and pestle, then passed through a 30 mesh screen by hand.

(8) Items 8-12 (Emcocel, ProSolv 50, ProSolv 90, LH-11, VivaSol) were de-lumped by passing through a 25-mesh screen into a polyethylene bag and manually shaken 30-times and added to the famotidine granules and mixed in an 8 qt. V-blender for 5 min. This produced Mixture 1.

(9) Items 6 and 7 (ibuprofen and colloidal silicon dioxide) were mixed and passed through a 25-mesh screen into a polyethylene bag. This produced Mixture 2.

(10) Mixture 1 and an equal amount of Mixture 2 were combined and mixed in an 8 qt. V-blender for 10 minutes.

(11) The remaining Mixture 2 was added and the combined material (“Mixture 3”) was mixed in an 1 cubic-foot V-blender for 10 minutes.

(12) Item 13 was added and the resulting mixture mixed in an 1 cubic-foot V-blender for 3 minutes.

(13) The formulation was pressed into tablets.

Tablets made with this formulation and method had the following properties: Content Uniformity (n=10): mean (ibuprofen 102.3%, famotidine 101.4%), RSD (ibuprofen 2.6%, famotidine 1.9%), meeting USP requirements. Dissolution: at least 95% released for both drugs after 30 minutes (measured under neutral assay conditions).

All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

This application is related to U.S. patent application Ser. No. 11/______, filed Jul. 18, 2006, entitled “Unit Dose Form With Ibuprofen-Famotidine Admixture” [Attorney Docket No. 026057-000800], the contents of which is hereby incorporated by reference in their entirety and for all purposes.

1. A method for administration of ibuprofen to a subject in need of ibuprofen treatment comprising administering an oral dosage form comprising a therapeutically effective amount of ibuprofen and a therapeutically effective amount of famotidine, wherein the ibuprofen and the famotidine are combined in an admixture with at least one excipient and wherein the oral dosage form is administered three times per day (TID).

2. The method of claim 1 wherein the famotidine and ibuprofen are released from the dosage form rapidly in an aqueous environment.

3. The method of claim 2 wherein said TID administration of said dosage form provides better gastric protection over a 24-hour period than TID administration of the same daily quantity of ibuprofen and two times a day (BID) administration of the same daily quantity of famotidine.

4. The method of claim 3 wherein the daily quantity of ibuprofen is about 2400 mg and the daily quantity of famotidine is about 80 mg.

5. The method of claim 4 wherein TID administration of a dosage form containing 800 mg ibuprofen and 26.6 mg famotidine provides better gastric protection over a 24-hour period than TID administration of the 800 mg ibuprofen and BID administration of 40 mg famotidine.

6. The method of claim 3 wherein the subject's intragastric pH is greater than 3.5 for at least 18 hours of a 24 hour dosing cycle.

7. The method of claim 6 wherein the subject's intragastric pH is greater than 3.5 for at least 20 hours of a 24 hour dosing cycle.

8. The method of claim 1 wherein the oral dosage form comprises ibuprofen and famotidine in a ratio in the range of 29:1 to 32:1.

9. The method of claim 8 wherein the oral dosage form comprises ibuprofen and famotidine in a ratio in the range of 30:1 to 31:1.

10. The method of claim 8 wherein the oral dosage form comprises about 750 mg to 850 mg ibuprofen and about 24 mg to 28 mg famotidine.

11. The method of claim 8 wherein the oral dosage form comprises about 375 mg to about 425 mg ibuprofen and about 12 mg to 14 mg famotidine.

12. The method of claim 1 wherein the oral dosage form comprises ibuprofen and famotidine in a ratio in the range of 20:1 to 25:1.

13. The method of claim 12 wherein the oral dosage form comprises ibuprofen and famotidine in a ratio in the range of 22:1 to 23:1.

14. The method of claim 1 wherein the subject is in need of ibuprofen treatment for a chronic condition.

15. The method of claim 14 wherein the chronic condition is rheumatoid arthritis, osteoarthritis or chronic pain.

16. The method of claim 1 wherein the subject is in need of ibuprofen treatment for acute pain, dysmenorrhea or acute inflammation.

17. The method of claim 4 wherein each dosage form comprises about 400 mg ibuprofen and about 13.3 mg famotidine.

18. The method of claim 4 wherein each dosage form comprises about 800 mg ibuprofen and about 26.6 mg famotidine.

19. The method of claim 1 wherein each dosage form comprises about 600 mg ibuprofen and about 26.6 mg famotidine.

20. A solid oral dosage form comprising a therapeutically effective amount of ibuprofen and a therapeutically effective amount of famotidine, wherein the ibuprofen and the famotidine are combined in an admixture with at least one excipient, wherein in an aqueous environment the ibuprofen and famotidine are released into solution rapidly and wherein the oral dosage form comprises famotidine in the range of 24 mg to 28 mg or in the range 12 mg to 14 mg.

21. The oral dosage form of claim 20 that contains about 13.3 mg famotidine or about 26.6 mg famotidine.

22. The oral dosage form of claim 20 that comprises ibuprofen and famotidine in a ratio in the range of 29:1 to 32:1 or 22:1 to 23:1.

23. The oral dosage form of claim 1 that comprises
from 750 mg to 850 mg ibuprofen and 24 mg to 28 mg famotidine; or,
from 575 mg to 625 mg ibuprofen and 24 mg to 28 mg famotidine; or
from 375 mg to 425 mg ibuprofen and 12 mg to 14 mg famotidine; or,
from 175 mg to 225 mg ibuprofen and 6 mg to 7 mg famotidine.

24. The oral dosage form of claim 23 that comprises
about 800 mg ibuprofen and about 26.6 mg famotidine or
about 600 mg ibuprofen and about 26.6 mg famotidine or
about 400 mg ibuprofen and about 13.3 mg famotidine or
about 200 mg ibuprofen and about 6.6 mg famotidine.

25. The oral dosage form of claim 23 wherein at least 75% of the famotidine and at least 75% of the ibuprofen in the dosage form are released within 15 minutes when measured in a Type II dissolution apparatus (paddles) according to the U.S. Pharmacopoeia at 37° C. in 50 mM potassium phosphate buffer, pH 7.2 at 50 rotations per minute.

26. The oral dosage form of claim 25 that is a tablet.

27. A method of treating a patient in need of ibuprofen treatment, where the patient is at elevated risk for developing an NSAID-induced ulcer, comprising administering an oral dosage form of claim 1.

28. A method of reducing symptoms of dyspepsia in a subject in need of NSAID treatment who has experienced symptoms of dyspepsia associated with NSAID administration, comprising administering to the subject an effective amount of a NSAID in combination with an effective amount of famotidine, wherein the famotidine is administered three times per day.

29. The method of claim 28 wherein the NSAID is ibuprofen.

30. The method of claim 28 wherein from 25 mg to 27 mg famotidine is administered three times per day.
